(19) United States
(12) Patent Application Publication
(10) Pub. No.: US 2003/0084914 A1
(43) Pub. Date: May 8, 2003

(54) Cosmetic articles having encapsulated liquid and method of making same

(75) Inventor: Pascal Simon, Vitry Sur Seine (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 09/986,264

(22) Filed: Nov. 8, 2001

(51) Int. Cl. A61K 9/48; A61K 7/00

(52) U.S. Cl. 132/333; 424/401

(57) ABSTRACT

A personal care or cosmetic article is provided which includes a substrate within which is disposed at least one liquid containing breakable capsule. The article further includes a cosmetic composition which is wetted by liquid within the capsule upon breaking of the capsule. Additional capsules can be provided for wetting the cosmetic composition or, alternatively, one or more capsules can be provided with the same article to wet additional cosmetic compositions, so that multiple functions are performed by the same article.

### BACKGROUND OF THE INVENTION

1. Technical Field

The invention relates to cosmetic articles and methods of making cosmetic articles. More particularly, the invention relates to cosmetic articles in which a surfactant or other cosmetic ingredients are present in solid form. The article also includes a liquid disposed in one or more breakable capsules such that, when the capsule or capsules are broken, the liquid wets the surfactant or other cosmetic ingredients.

2. Discussion Of The Background

Personal cleansing articles have been known for many years. Generally, there have been two types of such articles, dry wipes and wet wipes. For example, expired U.S. Pat. No. 4,303,543 to Mansy discloses impregnating a nonwoven cloth substrate with an aqueous solution of a surfactant and a conditioner. The nonwoven cloth substrate is then dried to form a “dry wipe.” When it is desired to use a dry wipe, the user simply wets the substrate with water, and proceeds to cleanse and condition the skin with the wipe. Such dry wipe articles have a number of shortcomings. Although these articles are supposed to be convenient to use, dry wipes can be inconvenient because they require a source of water to wet the dried surfactant so that the article can be used to cleanse the skin. Accordingly, the user must find a sink or bathroom facility in order to use the dry wipe. This requirement is inconvenient for travel or other situations in which restrooms are unavailable or unpleasant, or where the water quality is questionable. Dry wipes can also perform less than optimally if the user does not properly wet the dry wipe. For example, if the user holds the dry wipe in a stream of water for an excessive amount of time, the surfactant or other ingredients can be washed from the dry wipe before it is used. In addition, if an insufficient amount of water is added, insufficient lathering/foaming can result, and the wipe can also feel rough or abrasive, resulting in an unpleasant experience to the user.

As the name implies, wet wipes are packaged in a wet form, and therefore, avoid the need to add moisture for use. However, often the water or other liquid which wets the wet wipe will settle within the packaging, such that portions of the wet wipe become dry, resulting in poor performance of the wipe. In addition, wet wipes can be susceptible to bacterial or fungal growth. Further, if the wet wipes are packaged as a group within a container, once the container is opened, the liquid in the wet wipes evaporates and the product deteriorates. Moreover, wet wipes are usually formed of thin substrate materials, which are difficult to use to fully cleanse and scrub the skin, and the wipes are perceived as being of low quality. Also, wet wipe articles can be undesirably limited in terms of the compositions which can be used in the wipes, because certain compositions deteriorate when stored in wet form.

### SUMMARY OF THE INVENTION

The invention eliminates the shortcomings with prior art cosmetic and personal cleansing articles. The invention also provides an article which can be advantageously used for various cosmetics in addition to cleansing compositions. For example, the article could be used to apply a make-up composition such as a foundation make-up, to apply a skin care composition, a hair care product (e.g., shampoo, conditioner, and/or dye), a self-tanning product, a make-up removal product, or to apply/use other cosmetic compositions. The invention provides a wipe that includes encapsulated water and a cosmetic composition in solid form. When it is desired to use the wipe, the water capsule(s) is broken such that the cosmetic composition is wetted or dissolved by the water and the wipe is ready for use. This arrangement provides a number of advantages as compared with prior wet and dry wipes. For example, because the water is associated with the substrate, the user need not have access to a water source, and the articles can be conveniently used anywhere. In addition, because the water is encapsulated within the substrate, the amount of water is controlled, such that it better matches the amount of surfactant (or other cosmetic composition) associated with the substrate. Moreover, by associating a predetermined amount of water with the substrate, the user does not inadvertently wash away the ingredients of the substrate, as could have been the case with prior dry wipes if held under a faucet for an excessive period of time. Further, because the water is encapsulated, the wipes are not subject to drying as has been the case with wet wipes. Also, in contrast to typical wet wipes, the use of encapsulated water allows the convenient use of thicker substrates and substrates that expand when wet to provide a more pleasant experience to the consumer. In addition, as will be apparent herein, the encapsulated water allows the article to have multiple uses, for example, to apply two or more different products with the same article.

For most applications, the cosmetic composition is wetted by encapsulated water associated with the substrate. However, other encapsulated liquids could be provided instead of or in addition to encapsulated water. The article could have one or more oil containing capsules, for example, where an oil soluble conditioner or sunscreen is to be provided, or where it is desirable to encapsulate one of the cosmetic ingredients in one or more capsules.

The articles of the invention can include various types of ingredients in solid form. For example, the wipe can include a surfactant, a conditioner, vitamins (such as vitamin C or vitamin E), anti-wrinkle or anti-acne ingredients, fragrances, sunscreens, self-tanning compositions, make-up, make-up remover, shampoo, hair dye, hair conditioner, or other cosmetic ingredients. These ingredients can also be present in dry form, and are wetted or dissolved when the liquid capsule or capsules are broken. As will be apparent from the detailed description herein, the other ingredients can be mixed together. Alternately, one portion of the article can be utilized for the application of one composition, and another portion can be utilized for the application of another composition. In one example, the article can have a wet side and a dry side, such that the wet side is used to cleanse and/or condition the skin upon breaking of the encapsulated water, and the dry side is used to dry the skin after cleansing/conditioning.

The invention also provides advantageous packaging for the cosmetic articles as well as methods for manufacturing the articles.

### BRIEF DESCRIPTION OF THE DRAWINGS

A better appreciation of the invention will become further apparent from the detailed description provided herein, particularly when considered in conjunction with the accompanying drawings in which:

FIGS. 1-3 are cross-sectional views of different embodiments of cosmetic articles in accordance with the invention;

FIG. 4 depicts a substrate which can be utilized in accordance with the invention, and which can include one or more substrates webs, which can then be formed into articles according to the invention;

FIGS. 5a-5j depict an end view of examples of various substrates which can include one or more substrate webs, and which can be utilized in the articles of the invention;

FIG. 6 depicts an example of an apparatus which can be utilized in accordance with one method of forming articles in accordance with the invention;

FIGS. 7A and 7B depict alternate apparatus and methods for forming the articles according to the invention;

FIGS. 8a-8c depict an end views of examples of various alternative substrates that can be utilized in accordance with the invention;

FIG. 9 depicts a cross-sectional view of an alternate embodiment of an article of the invention;

FIG. 10 depicts an example of another alternate apparatus and method for forming articles according to the invention;

FIGS. 11A and 11B depict cross-sectional views of alternate embodiments of cosmetic articles according to the invention;

FIGS. 11C and 11D depict plan views of alternate embodiments of cosmetic articles according to the invention; and

FIGS. 12 and 13 depict different containers which can be utilized for packaging/storing articles of the invention, and which also include means for taking the capsule(s) in cosmetic articles according to the invention.

### DETAILED DESCRIPTION OF THE PREFERRED EMBODIMENTS

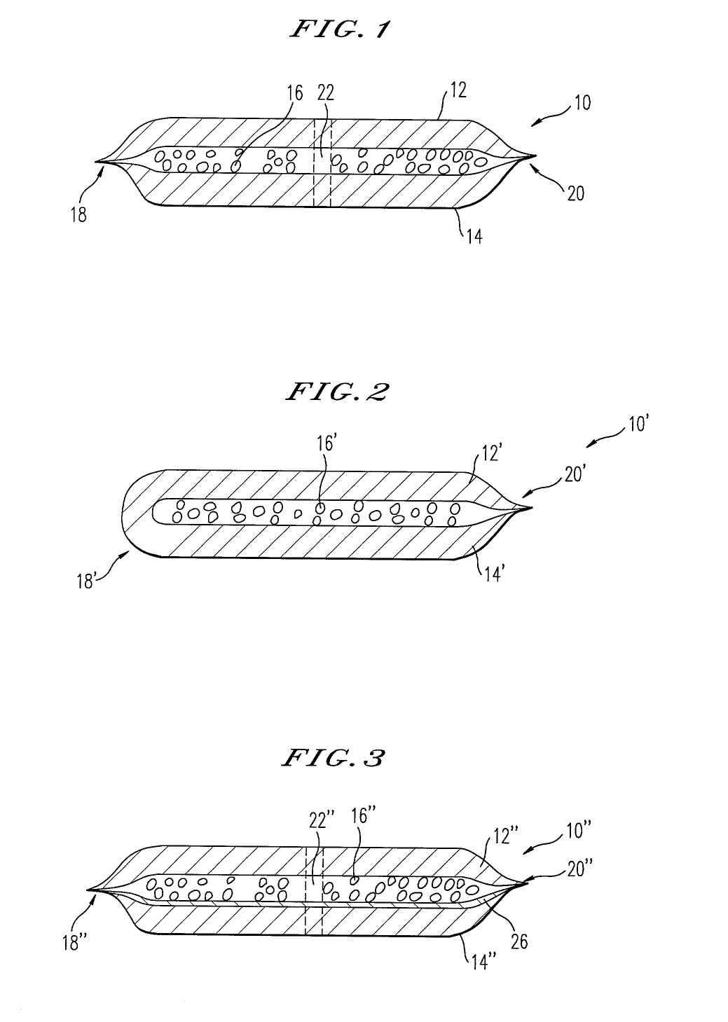

FIG. 1 depicts, in cross-section, an embodiment of the invention in which the article 10 includes a substrate having a first layer 12 and a second layer 14. A plurality of water-filled capsules 16 are disposed between the layers 12, 14 such that water is held between the substrate layers 12, 14, but the substrate layers themselves can be kept dry until use is desired. A cosmetic composition can be associated with the substrate by disposing the composition in the region in which the encapsulated water is disposed, i.e., between the layers 12, 14. This can be achieved by providing the composition in powder, particulate or granular form in the region between the layers 12, 14, or by providing the composition as a coating on the capsules 16. Alternately, one or both of the layers 12, 14 can include a cosmetic composition associated therewith. For example, a surfactant (or other composition) can be added to one or both of the layers 12, 14 in an aqueous form, with the layer(s) then dried before being assembled to form the article 10. The composition could also be added to the substrate as a hot coating, which becomes solid upon cooling. As a further alternative, one or both of the layers 12, 14 can be formed of at least two plies, with the composition sandwiched between the plies. By way of example, to manufacture a product in which the surfactant is sandwiched between plies, the surfactant and/or other ingredients are provided in powder form and mixed with an adhesive that is also in powder form. Alternately, the cosmetic composition and adhesive could be applied separately, without prior mixing. The powders are deposited upon a first substrate ply, a second substrate ply is overlaid upon the first ply, and the plies are heated to melt the adhesive. Upon cooling of the adhesive, the plies are bonded together with the surfactant sandwiched therebetween. This two-ply layer can then be used for one or both of the layers 12, 14 of the article 10.

The layers 12, 14 are fastened at their edges as indicated at 18, 20. Preferably, the layers 12, 14 are bonded utilizing heat and/or an adhesive. Bonding using heat “and/or” an adhesive can include fastening of the layers by, for example, bonding using a heat activated adhesive, bonding using an adhesive which can cure with or without requiring the application of heat, and bonding by heat where the heat causes the substrate plies or layers to be fused or welded together. However, other fastening expedients could also be utilized, such as stitching, fastening of the webs by crimping and/or impailing of the webs to lock the webs together, etc. Optionally, the layers 12, 14 can be fastened or coupled at one or more additional locations between the side or peripheral edges of the article as shown at 22. This additional fastening provides a location at which the article can be folded, for example, so that the article can be packaged in a C-fold or U-fold condition (or other fold configurations). As with the edges 18, 20, the layers can be fastened at 22 by an adhesive and/or heat, by stitching, or other fastening expedients. This additional coupling divides the water capsules 16 at a location to be folded so that capsules are not broken upon folding. Even if the product is not folded, the separation at 22 can also be advantageous to separate different portions of the substrate if it is desired to use the different portions for different uses or different cosmetic compositions as discussed further hereinafter.

Although the layers 12, 14 are provided as two separate webs in FIG. 1, they could also be formed from a single web, with the layers delimited by a fold 18′ at one side as shown in the FIG. 2 article 10′. The layers 12′, 14′ are fastened at the edge opposite the fold 18′ as shown at 20′. Even if the layers are coupled by a fold 18′ at one edge of the article, that edge could additionally include a seam (formed, for example, by heating or stitching in the region of the fold 18′) to fasten the layers so that the edge 18′ appears similar to edge 20′ and the article has a more uniform appearance. The layers 12, 14 or 12′, 14′ are also attached at edges not shown (considering the page of the drawing as a cross-sectional plane through the article, an additional edge would be in front of the plane and another edge behind the plane) in the figures to define an envelope or pocket which contains the water capsule or capsules.

The arrangement of FIG. 1, in which the layers 12, 14 are formed of separate webs, is particularly advantageous if it is desired to have different properties (texture, roughness, softness, permeability, etc.) for the different layers 12, 14. With different types of webs for layers 12, 14, the user has the option to utilize the layer most comfortable, or to alternate layers during cleansing if desired. In addition, one layer could be selected as more suitable for sensitive skin areas such as the face, while the other layer is more suitable for other areas of the body. Also, one layer 12 could be permeable so that it can be used for cleansing (or application of another cosmetic composition), while the other layer 14 is impermeable or less permeable to prevent the loss of liquid through the back or non-applicator side of the article.

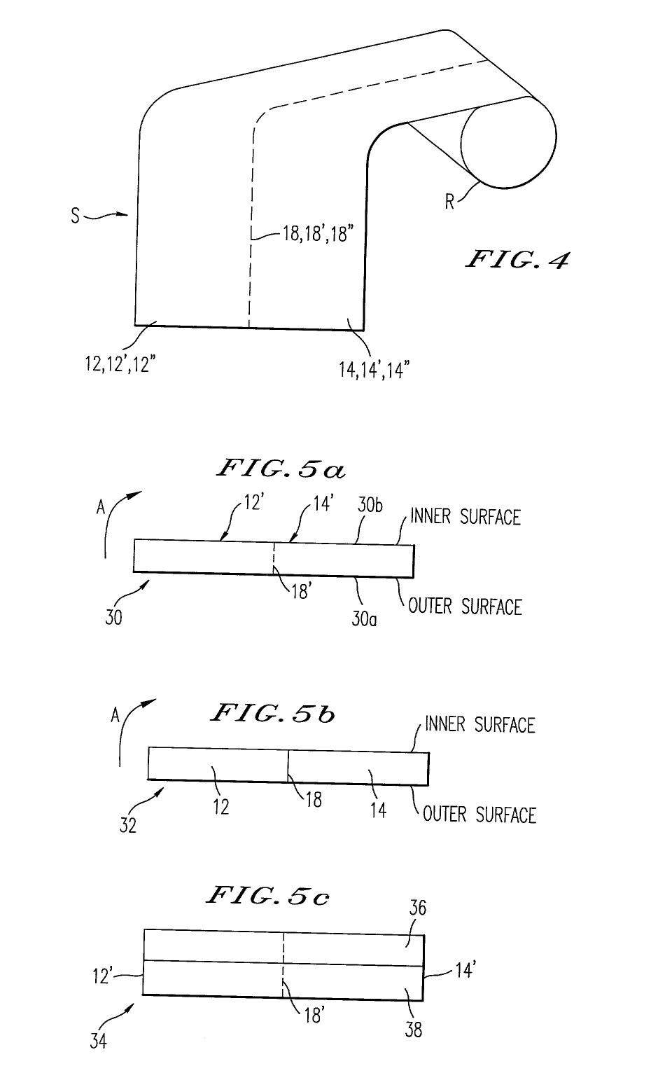

FIG. 3 depicts another alternate embodiment of the invention. The article 10″ of the FIG. 3 embodiment includes a moisture impervious ply or moisture resistant ply 26 associated with the layer 14″. With this embodiment, when the capsules 16″ are broken, the layer 12″ (and the surfactant or other ingredients associated with the article 10″) will become wet, while the layer 14″ remains dry. Thus, the user will have a dry side to hold while cleansing with the wet side 12″, and thereafter, the dry side 14″ can be used to wipe and dry the area cleansed. As in the embodiments of FIGS. 1 and 2, the layers 12″, 14″ can be joined at edge 20″. The other edge 18″ can be fastened and/or the layers can be connected at a fold (if the article with the layers 12″, 14″ have a common web as in the FIG. 2 embodiment). If the layers 12″, 14″ of the article 10″ are formed by folding a web (at 18″), the moisture impervious or moisture resistant layer 26 can be applied to one part of the web which is to become the layer 14″ as discussed in further detail hereinafter.

As with the earlier embodiments, the FIG. 3 embodiment can optionally include an additional coupling 22″ between the layers 12″, 14″. As noted earlier, this additional fastening or coupling can provide a location at which the article can be folded without breaking the capsules. In addition, or alternatively, the coupling 22″ can act as a divider to reduce mixing of the liquid and/or composition in one region of the substrate layer 12″ with the liquid and/or composition in the remainder of that layer. This feature can be particularly advantageous in providing a wipe which has different functions for different portions of the wipe. For example, the wipe of FIG. 3 can include a surfactant provided in the portion of the substrate layer 12″ to the right of the divider 22″, with no surfactant present in the region to the left of the divider 22″. As a result, the wipe can be formed to have at least three functions. The portion to the right of the divider 22″ of layer 12″ can be used for washing or cleansing. The cleansed skin area can then be rinsed using the portion of the substrate layer 12″ that is to the left of the divider 22″. After washing and rinsing, the user can dry the skin using the substrate layer 14″ on the reverse side of the substrate. As should be readily apparent, a number of variations of this multi-function wipe are possible. For example, the rinse portion of the substrate could be free of any cosmetic composition so that it only provides a liquid such as water for rinsing. Alternately, the rinse portion of the substrates could additionally include a conditioner, a sunscreen, vitamins or other cosmetic composition ingredients associated therewith. Alternately, the two portions of the layer 12″ can be utilized for two different cosmetic compositions, e.g., one portion for make-up remover and another for a conditioner, or with one or both portions utilized for application of make-up compositions.

The provision of separate regions for different cosmetic compositions can be desirable in more reliably applying the compositions to the skin as compared with, e.g., a wipe in which different compositions (such as a surfactant and a conditioner) are provided in the same portion of the substrate. Because the ingredients can be applied separate from, e.g., a surfactant, the user can better control the amount of the additional ingredients to be applied (or the user can choose to not apply the additional ingredient or ingredients). For example, the FIG. 3 embodiment can be formed with a surfactant associated with the portion of the layer 12″ to the right of divider 22″ and a conditioner associated with the portion of layer 12″ to the left of the divider 22″. With this arrangement, the user can cleanse the face with the right portion of layer 12″ and then dry the face with layer 14″. Thereafter, the user can optionally apply a conditioner (or other composition) with the left portion of layer 12″ (or the user could apply the cosmetic composition of the left portion before drying, if desired). With this arrangement, the user can better control the application or use of the different compositions because, in applying the conditioner (or other composition), the user has the option to apply a light or heavy amount, or to apply the conditioner to only selected areas of dry skin. Moreover, because the conditioner is applied separately, it is not washed/wiped away when the user removes the surfactant from the skin. This ability to control the amount and/or selectively apply additional ingredients can be desirable not only with conditioners, but with other ingredients as well, for example anti-wrinkle or anti-acne ingredients tanning/sun screen products, make-up compositions etc. Of course, this arrangement (i.e., in which compositions of different sections are separated from each other) is also desirable where two compositions are incompatible with each other or where mixing is otherwise undesirable, e.g., with two make-up compositions.

With a multi-function wipe, various indicia can optionally be provided on the wipe. This indicia can be in the form of different colored layers or differently colored sections, printing, or other indicators which allow the user to readily identify the different sections or functions of the different regions. For example, if one side of the wipe is used for washing and the other for drying, one layer can be white and the other blue. In addition, or alternatively, indicia such as “cleanse” and “dry” could be provided on the respective substrate layers (e.g., by printing, a label, stitching or any other suitable forms or combinations of forms). Similarly, where three or more functions are provided, indicia such as “cleanse,” “rinse,” “dry,” “condition,” etc. can be provided in the different regions of the substrate. It should be noted that the provision of a fastener or divider in the region 22″ is not necessary to impart different functions to different portions of the layer 12″. For example, even without such a divider, the right portion of the substrate layer 12″ can be provided with a surfactant for cleansing, while the left portion can be used for rinsing or application of another ingredient and, because the different ingredients are associated with different sections of the substrate the amount of mixing is reduced. However, the use of an additional coupling or divider 22″ can be additionally advantageous to reduce the amount of mixing which could occur along the different portions of the substrate. Also, the seam or divider can allow the user to readily distinguish the different regions of the substrate.

Although the embodiments of FIGS. 1-3 include plural small capsules, it is to be understood that a single or smaller number of larger capsules could also be used. By way of example, in the FIG. 3 embodiment having a divider 22″, a single larger capsule could be provided to the right of the divider 22″ and another larger capsule to the left of divider 22″. As a further alternative, one or more capsules could be provided to one side of the divider 22″, with no liquid capsule to the left, in which case the left portion of the substrate can be used for wiping/drying, and/or the left portion can be used as a handle/holding region of the substrate which the user grasps while applying a cosmetic composition with the right side.

Referring to FIGS. 4-8, non-limiting examples of manufacturing methods which can be used to form articles of the invention will be described. It is to be understood that other manufacturing methods are also possible. Generally, two types of manufacturing methods are presently preferred to manufacture articles according to the invention. In one form, either a single substrate web is used, or various components webs of the substrate are coupled together to form a substrate web, and this web can then be formed into the shape of the article and filled with the liquid containing capsule(s) to provide the cosmetic article. Alternately, two or more substrate components can be fastened together when the capsules are deposited between the substrate layers with the substrate layers then fastened together to enclose the microcapsules of water.

Referring to FIGS. 4-6, the first general approach will be described. With this method, the substrate is first formed into a single substrate web “S” having the various substrate components fastened together as shown in FIG. 4. Where the article is formed of two webs, for example, to form the FIG. 1 embodiment, a web corresponding to the layer 12 and a web corresponding to layer 14, the webs are joined along seam 18, and this seam will become the edge 18 of the article as shown in FIG. 1. This composite substrate web is then formed into the article 10 with one or more water capsules disposed therein as discussed in further detail hereinafter.

As noted earlier, with the FIG. 1 article, one or both layers 12, 14 can have a surfactant or other ingredient associated therewith. Each layer 12, 14 can be formed as a single ply, and if ingredients or compositions are associated with that layer, they can be applied, for example, by dipping the layer in a solution and thereafter allowing the layer to dry. Alternately, one or both layers can be formed of multiple substrate plies, and the ingredient(s) can be applied to the layer by various methods such as by dipping or by sandwiching the ingredients between plies of that layer. The layers 12, 14 can be joined together before or after the surfactant and/or other compositions are associated therewith. For example, the first layer 12 can be formed as a two ply layer with the ingredient(s) or composition(s) sandwiched therebetween, and the layer 14 can be formed as a two ply layer with a composition (which can be the same as layer 12 or a different composition) sandwiched therebetween. The layers 12, 14 can then be joined along seam 18.

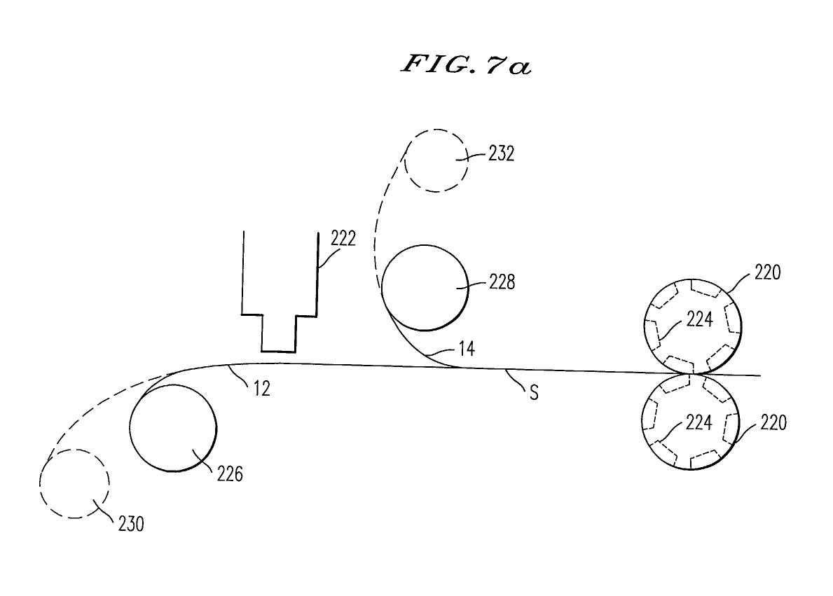

Alternately, a first ply of layer 12 can be joined to a first ply of layer 14 to form a first composite ply, which will become the outermost plies of the article of FIG. 1. The composition(s) can then be sandwiched between this composite ply and another ply (which will be disposed interiorly of the outer plies in the completed product) to form the substrate S, or the composition can be applied to one or more of the plies, e.g., by dipping or spraying the ply or plies with a solution containing the composition(s). The inner ply can be formed of a material which is either advantageous from a cost standpoint (because this inner ply will not be in contact with the skin, less expensive materials could be used) or could be formed of a material which is desirable from a standpoint of thickness, permeability and/or absorbency. This inner ply web can be the same for both layers 12, 14. If desired, the inner ply web could also be formed as a composite ply of two different ply materials joined at a seam, to allow the inner plies to impart, for example, different thickness, permeability or absorbency characteristics to the layers. Thus, to form the FIG. 3 embodiment, in which the layer 14″ has a liquid impervious layer 26 associated therewith, the impervious ply can be fastened/bonded to a permeable ply to form a composite inner ply for the substrate (part of which is permeable, the other of which is impermeable or moisture resistant), and this composite inner ply can be joined to an outer ply which can be a single web ply or composite ply (i.e., two or more webs coupled together). Alternately, the liquid impervious ply can be joined to the outer ply of layer 14 before the layer 14 is joined to the layer 12. Once the substrate web S is formed, the substrate web S is folded, filled, and sealed to provide the completed article. The substrate web S can be formed and stored as a stock supply or roll R, which is then formed into the article or the substrate components (if there are multiple components) can be coupled together upstream of a form/fill/seal type apparatus (discussed hereinafter with reference to FIG. 6) such that the web components are joined together in-line with the forming of the articles and insertion of the liquid capsule(s).

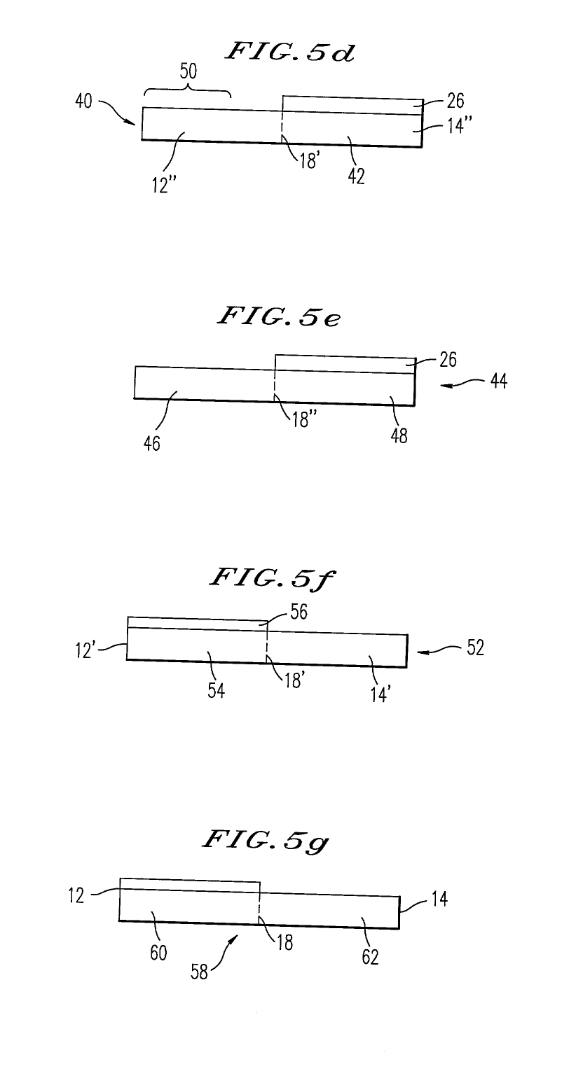

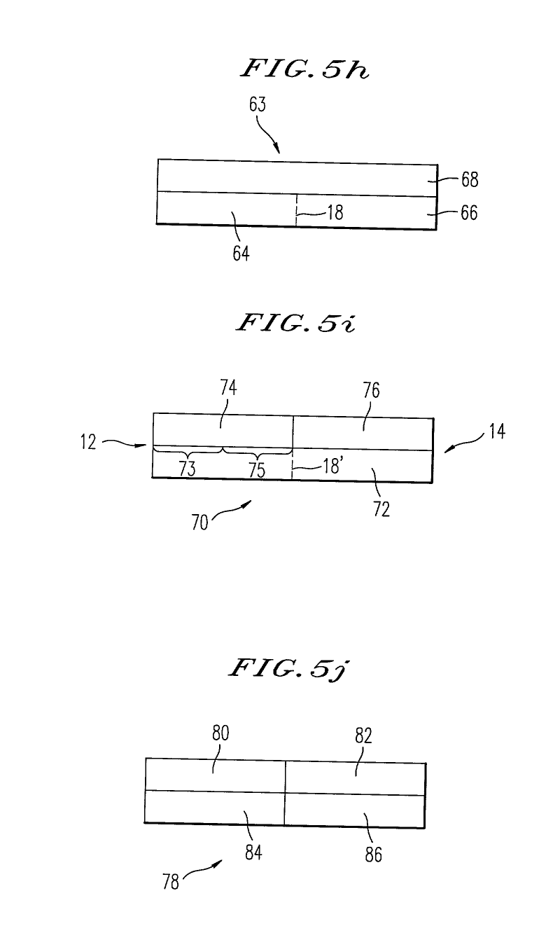

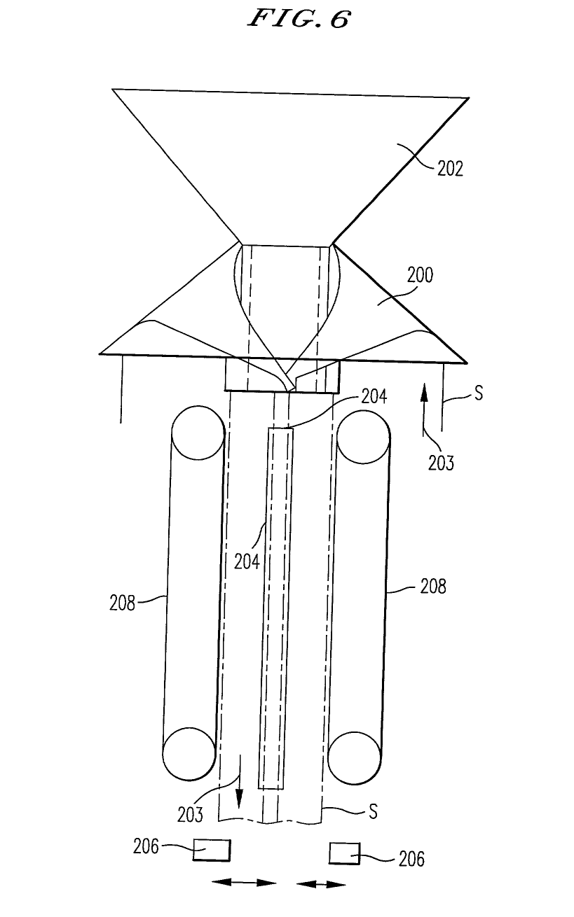

As should be apparent, the substrate web S can take various forms. FIGS. 5a-5j depict various nonlimiting examples of substrates in which the substrate web S is formed prior to filling with one or more liquid capsules. FIGS. 5a-5j are schematic block representations of an end view of various examples of substrates. The figures are not intended to reflect relative dimensions/thicknesses, nor are they intended to reflect precise locations at which the various webs/web components are joined. For example, some of the embodiments depict webs abutting each other, while in actuality, the webs would typically have a small overlap at which they can be fastened together. For the substrates of FIGS. 5a-j, the left side of the substrate will become the layer 12, 12′, or 12″, while the right side of the substrate will become the layer 14, 14′, or 14″. Also, the top of each drawing will be the inner or interior portion of the substrate—the portion which will be adjacent to the liquid containing capsule or capsules. The bottom part of the substrate will become the outer portion of the substrate—the part which contacts the skin during use.

FIG. 5a depicts a simple form of the substrate in which a single ply substrate web 30 forms both layers 12′, 14′. This ply can then be folded (as represented by arrow A), filled with one or more liquid containing capsules, and the edges fastened to form an article as shown in FIG. 2. The region indicated at 18′ then becomes the left edge of the article of FIG. 2, the surface 30b becomes the inner surface of the substrate (adjacent to the liquid capsules), and the surface 30a becomes the outer surface of the article.

FIG. 5b depicts a substrate web 32 in which the upper and lower layers 12, 14 are different, and attached by a seam 18. This arrangement allows the layers 12, 14 to have different properties, for example, different textures or roughnesses as discussed earlier. As with the FIG. 5a embodiment and the embodiments of FIGS. 5c-j discussed below, after the substrate web 32 is formed, the substrate can then be formed into the finished article by folding the web in the direction of arrow A, filling the substrate with one or more liquid containing capsules, and sealing the edges of the substrate.

The layers of the substrates of FIGS. 5a and 5b are each single ply layers, and therefore, the sandwiching method of associating the surfactant (or other ingredients) with the substrate is not utilized, and other methods of applying the surfactant to the substrate are used. For example, the surfactant could be applied to one or both of the layers 12, 14 in an aqueous or other liquid form (e.g., a hot melt), and the substrate thereafter dried or cooled. The surfactant or other ingredients could also be added at the same time the substrate is filled with liquid containing capsules so that the surfactant is disposed between the layers 12, 14.

The FIG. 5c substrate web 34 is a two ply substrate. For multiple ply substrates, the cosmetic composition(s) can be associated with the substrate by the sandwiching method, in which the surfactant (or other cosmetic composition) is sandwiched between the plies. Where one or both of the layers 12, 14 are formed of two or more plies, other methods could also be utilized to apply the composition(s) to the substrate, such as dipping or spraying a solution containing the composition, or applying a hot melt containing the composition, etc. The use of two plies for one or more of the layers 12, 14 can also impart advantageous performance characteristics to the article. For example, the outer ply 38 can be a ply selected as best suitable for contact with the skin, while the inner ply 36 can be selected for its moisture absorption or permeability characteristics. By way of example, if the outer ply 38 is a relatively open or porous material and the substrate is a single ply substrate, upon breaking of the water capsules, the water could simply pass through the substrate and be ineffective in cleansing the skin, promoting lathering of a surfactant, or otherwise assisting the wetting or use of other cosmetic compositions. By contrast, if an absorbent or moisture retaining inner ply 36 is provided, this ply can retain the moisture after the water capsules are broken so that the moisture can be utilized for wetting and application of a cosmetic composition and/or rinsing. Thus, for example, the layer 36 could be a layer which has superior moisture retaining qualities as compared with layer 38, while the layer 38 has properties desirable for contacting the skin. The substrate web 34 can be folded (at 18′) to form an article as shown in FIG. 2. With this arrangement, the layers 12′, 14′ will have a common inner ply 36 and a common outer ply 38. A surfactant (or other cosmetic composition) and an adhesive can be sandwiched between the plies of each of the layers 12′, 14′. The surfactant or other composition can be applied over the entire substrate, it could be provided between plies of only one layer, or at only selected portions of one or both layers. Also, more than one cosmetic composition could be applied, for example, with one composition applied to layer 12′ and another applied to layer 14′. The adhesive should be present over a sufficient area between the plies to ensure a good bond between the plies.

FIG. 5d depicts a substrate web 40 that can be utilized to form an article as shown in FIG. 3. With this substrate web 40, an impervious or moisture resistant ply 26 is applied to a web 42. The web 42 will form the outer ply for both layers 12″ and 14″. With this substrate web 40, when the article is formed, a moisture barrier is provided between the liquid capsule and the layer 14″ so that the layer 12″ can be used for cleansing and/or conditioning (or application of other cosmetic compositions), while the layer 14″ can be used for wiping or drying the skin. The ply 26 could be a web formed before it is applied to the remainder of the layer 14″. Alternately, the ply 26 could be formed by distributing an impervious or moisture resistant coating upon the portion of the web 42 which will become the layer 14. The layer 12″ will have the surfactant or other ingredients associated therewith, for example, by applying the surfactant as a liquid and then drying the substrate or by applying the surfactant as a hot coating which cools to a dry solid at room temperature. Alternately, the surfactant or other ingredients can be disposed in the region between the layers 12″, 14″ as discussed earlier. If it is desired to form a wash, rinse, and dry article as discussed earlier, the surfactant can be provided on only a portion of the substrate layer 12″. For example, if a surfactant is applied only to the region 50 of FIG. 5d, this region becomes the region of layer 12″ to the right of the fastener/divider location 22″ of FIG. 3 and is used for cleansing. The remainder of the layer 12″ can then be used for rinsing or the application of other ingredients, and layer 14″ can be used for drying. With the FIG. 5d arrangement, the layers 12″, 14″ are formed from a common web 42, and can be folded about 18′ to form an article with the capsule(s) between the layers as discussed earlier with reference to FIG. 3.

The substrate web 44 of FIG. 5e is similar to FIG. 5d, except that the outer ply of the article is formed of two webs 46, 48 joined along seam 18″. As discussed earlier, this allows the surfaces of the layers 12″, 14″ to have different properties (textures, roughness, softness, absorbency, etc.). The ply 26 can be fastened to the ply 48, before, during, or after coupling of the ply 48 to the ply 46. As with the embodiment of FIG. 5d, the surfactant can be applied over all or only part of the ply 46 (which becomes the layer 12″). As noted above, the ply 26 can be impermeable or less permeable than the substrate webs 46 and/or 48. Alternately, the ply 26 could be provided as a ply having better moisture retention characteristics than web 46 and/or 48 (12″ and/or 14″ in the FIG. 5d embodiment), to assist in retaining the liquid in the substrate upon breaking of the capsule. Also, the ply 26 could be used to retain ingredients of the article between ply 26 and the web 48 (or 42 in FIG. 5d).

FIG. 5f depicts a substrate web 52 which can be used, for example, in forming the FIG. 2 embodiment. With the FIG. 5f substrate web 52, the surfactant or other composition can be associated the layer 12′, e.g., utilizing the sandwiching method. As a result, the layer 12′ will have two plies 54, 56, between which the surfactant or other cosmetic composition is sandwiched as discussed earlier. The layer 14′ is a single ply, and is formed of a web 54 which is common to the layer 12′. Thus, the outer surface of the layers 12′, 14′ (the surface exposed to the skin) will be the same.

The substrate web 58 of FIG. 5g is similar to FIG. 5f, except that two webs 60, 62 form the outermost plies for the layers 12, 14. Accordingly, this article can have the surfactant (or another cosmetic composition) associated with one of the layers 12, and that layer is a multiply layer. The webs 60, 62 are fastened as represented at 18. With this arrangement, the layer 14 can have different properties (e.g., roughness, softness, absorbency, permeability, etc.) as compared with the layer 12.

The substrate web 63 of FIG. 5h is similar to that of FIG. 5c, except that the outermost plies of the layers 12, 14 are formed of two webs 64, 66. The webs 64, 66 can be different so that the outermost ply of the layers 12, 14 can have different properties, e.g., different textures, absorbencies, permeabilities. As with the FIG. 5c embodiment, this substrate can have a cosmetic composition sandwiched between the inner ply 68 and one or both of the outer plies 64, 66. This can be accomplished by either applying the composition and the adhesive to the inner ply 68 and then placing the composite outer ply (64, 66) thereover, or by placing the cosmetic composition and adhesive upon the composite outer ply (64, 66) and then placing the inner ply 68 thereover. If the surfactant or other cosmetic composition and adhesive are applied to the inner ply, the webs of the outer ply could then be laid upon the inner ply and bonded to the inner ply without additional fastening of the outer plies together, because the outer plies 64, 66 will be bonded together by way of the inner ply 68. Alternately, the outer plies 64, 66 could be fastened together at 18 to form a composite outer ply web and this composite outer ply web can then be bonded to the inner ply web 68 to sandwich a surfactant or other cosmetic composition therebetween. Of course, the composition(s) could be applied to one or more of the plies 64, 66, 68 by other methods such as dipping or spraying with a solution containing the composition, with the play or plies then dried.

FIG. 5i depicts a substrate web 70 in which the layers 12, 14 have outer plies formed of a common web 72 but have different inner plies 74, 76. With this arrangement, the outer plies of the layers 12, 14 will, of course, have the same properties. The inner plies 74, 76 can have different properties, for example, the ply for layer 12 can be an absorbent liquid permeable ply, while the ply for the layer 14 can be an impermeable or moisture resistant ply. A surfactant or other composition can be sandwiched between the inner ply 74 and the outer ply 72 for the layer 12, while the inner and outer plies for the layer 14 are bonded without a surfactant therebetween. Accordingly, this embodiment can provide an article as described earlier with reference to FIG. 3, in which one side of the article is wet and can be used for cleansing with a surfactant or application of another cosmetic composition, while the other side is dry and can be used for wiping or drying. In addition, by providing a surfactant in only a portion of the sandwiched region between plies 72 and 74, a wash, rinse and dry article can be provided. For example, the region 73 can include an adhesive and surfactant, with the region 75 including the adhesive but no surfactant, to provide a wash, rinse, and dry article as discussed earlier. Alternately, a conditioner and adhesive can be applied to the region 75 to provide a wash (region 73 of layer 12), dry (layer 14), and conditioner (region 75 of layer 12) article. Of course, a wide variety of combinations are possible with this article. For example, in lieu of (or in addition to) a surfactant or conditioner composition in region 75, various other compositions could be provided, such as anti-wrinkle, anti-acne, sunscreen, make-up, hair care compositions, make-up remover, or vitamins (e.g., vitamin C or E).

The substrate web 78 of FIG. 5j is similar to that of FIG. 5i, except that, in addition to having different inner plies 80, 82, the article also has two different outer plies 84, 86. With this arrangement, the plies 84, 86 that are exposed to the skin can have the same or different properties (e.g., roughness, softness, permeability, and/or absorbency). The inner layers 80, 82 can also have different properties, e.g., with one ply absorbent and permeable and the other ply impermeable or less permeable.

FIG. 6 depicts a method and apparatus which can be utilized for forming articles in accordance with the invention. The equipment shown in FIG. 6 is generally known as a form, fill, and seal assembly. Similar equipment has been known for use in forming packaging. However, heretofore, there has been no recognition as to the use of such equipment to form cosmetic articles, particularly to form cosmetic articles containing one or more liquid containing capsules. Once the substrate web is formed by providing a substrate web, or by combining plural substrate webs as discussed above, the substrate is filled with one or more liquid containing capsules and the free edges of the substrate layers are fastened to each other to form the article.

The arrangement of FIG. 6 is particularly advantageous where the articles are to be formed of a single substrate S. This can be achieved where the article includes only a single substrate ply as shown in FIG. 5a, or where the substrate is formed of two or more plies which have been associated to form a single web as discussed earlier with reference to FIGS. 5b-5j. The substrate S can then be fed to a tube former 200, which forms the article into a tube so that an interior cavity is provided into which one or more liquid containing capsules can be introduced, for example, utilizing a funnel 202 or other suitable inserting mechanism. The substrate is fed in the direction shown by arrow 203 so that edges of the substrate S are brought together by the former 200, and the edges are then sealed by a suitable sealing mechanism 204 to form the edge 20, 20′, 20″, as shown, for example, in FIGS. 1-3. The seal mechanism can have various forms. For example, the seal mechanism 204 can provide a weld or heat seal, or the seal mechanism 204 can provide a stitch or adhesive or any other fastening to join the layers together. In addition to the longitudinal fastening of the edges together, the articles are sealed or seamed transversely with a transverse seal mechanism 206. As with the seal mechanism 204, the transverse seal mechanism 206 can take various forms including, for example, a welding, adhesive applicator, or a stitching mechanism. As shown in FIG. 6, two reciprocating heads 206 can be provided, with the reciprocating heads periodically moving toward one another to form a seal. Either a single pair of heads 206 or two pairs of heads can be provided. Where two pairs of heads are provided, one pair forms a seal at the bottom of an article while the other forms the seal at the top of an article. Alternately, a single pair of heads can form the seal for the top of an article at the same time it is forming the seal for the bottom of the next article, and the articles are separated by cutting through the seam or seal (so that part of the seal closes an end of one article and the rest of the seal closes an end of the next succeeding article). “Seal” is used herein in its broadest sense, and is not limited to hermetic seals, because the seal need not be impervious or hermetic, and can include various welding, adhesive, stitching, or other fastening expedients.

In operation, the transverse seal mechanism closes the bottom of the tube formed by the tube former 200 so that one or more liquid capsules can be introduced via funnel 202 without falling through the bottom of the tube. After introduction of the liquid capsule or capsules, the top transverse seam for that article is formed. As noted above, this can either be formed by the same pair of heads 206 in which case the heads 206 are forming the bottom seam of an article to be filled at the same time they are forming the top seal of an already filled article, or, alternately, separate heads can be provided for forming bottom seals and top seals for the articles. The substrate S can be conveyed by various mechanisms. For example, as shown in FIG. 6, a pair of conveyors 208 can be provided to feed the substrate S. Such conveyors can have a vacuum applied thereto to better hold the substrates, or they can urge the substrate against back-up supports disposed inside of the substrate to better grip the substrate. The heads 206 can also be mounted for vertical or rotary movement. For example, plural pairs of heads 206 can be mounted upon respective turrets so that the sealing operation is performed as the substrate is moving vertically downward. The vertical movement of the heads 206 can be desirable so that the forming, filling and sealing operations can occur while the substrate S is continuously moving. Alternately, the substrate S can be fed intermittently, so that the feed of the substrate S is halted during each transverse sealing operation. Where the heads 206 move vertically, for example, by a rotary mounting of the heads 206 upon turrets, the heads 206 can also serve to convey the substrate, since the successive of pairs of heads can continuously grasp the substrate and move the substrate downward. Thus, the heads 206 can also convey the substrate S as an alternative to the use of conveyors 208. The heads 206 can also include a cutting mechanism, such as a knife. Alternately, a separate cutting knife can be provided at a location downstream from the heads 206. By way of example, U.S. Pat. No. 3,557,525 depicts an arrangement in which moveable heads include a cutting knife. As noted earlier, form, fill and seal type arrangements have been known for many years. However, such arrangements have not heretofore been utilized for forming cosmetic articles.

In addition to utilizing the funnel 202 for introducing one or more liquid containing capsules into the substrate, the funnel 202 can also be utilized for introducing other ingredients of the article in addition to, or as an alternative to, associating ingredients with the substrate layers or plies (e.g., by previously dipping the substrate or one or more plies of the substrate into a solution including one or more of the ingredients, by sandwiching one or more ingredients between plies of the substrate, or by spraying a solution onto the substrate). Also, as noted earlier, ingredients could be associated with the one or more liquid capsules, for example, by coating the capsules with one or more ingredients. If it is desired to provide an additional separation (e.g., to form a separation as shown at 22″ in FIG. 3) or divider in the articles, the assembly of FIG. 6 can include an additional longitudinal seal mechanism to seal the substrate layers with this arrangement, two funnels or other inserting mechanisms 202 can be provided with the longitudinal seal mechanism positioned to form a seal between the funnels to form a divider as shown at 22″ in FIG. 3, with the funnels respectively depositing one or more liquid containing capsules on each side of the divider 22″. As a further alternative, in order to form a multi-compartment article, the substrate edges can be overlapped sufficiently so that two compartments are formed by the substrate web. By way of example, such an arrangement is shown in U.S. Pat. No. 3,795,081 which is incorporated herein by reference. Although the arrangement of FIG. 6 provides a vertical feed of the substrate, the substrate could also be fed horizontally, with the substrate folded over and the edges sealed/fastened together to enclose one or more of the liquid containing capsules such that the liquid containing capsule(s) is held within the substrate.

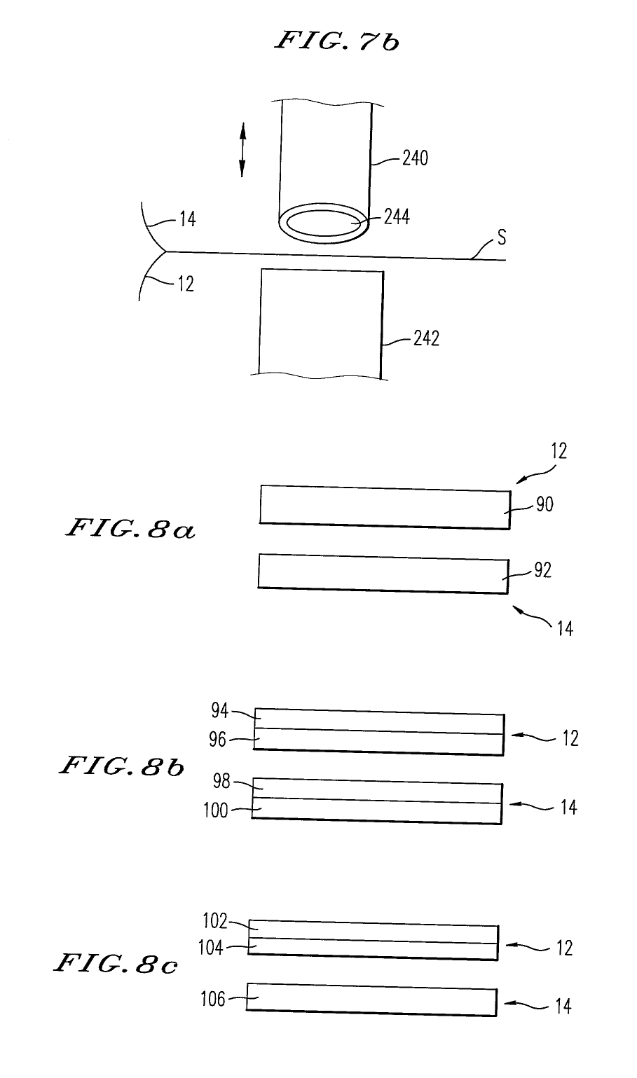

As noted above, as an alternative to utilizing a single substrate web S, in which the various component webs are joined to form a substrate web S before the substrate is filled and formed into the cleansing article, two or more webs can be joined during or after introduction of the capsules between the layers 12, 14. FIGS. 7A and 7B depict examples of apparatus in which two webs can be joined as the capsules are disposed between the layers.

The arrangement of FIG. 7A can be utilized where the various plies are not formed into a single substrate S before introduction of one or more of the liquid containing capsules. In the arrangement of FIG. 7A, the substrate layers are joined as they pass through a pair of nip rolls 220. The liquid containing capsule or capsules as well as other ingredients can be introduced onto one of the layers 12, after which another layer 14 is overlaid, with the layers 12, 14 joined together by the nip rolls 220. Preferably, one or both of the nip rolls include suitable pockets as shown in broken line at 224 so that the capsules are not broken during sealing. The pockets can have various shapes such as oval, circular, square, rectangular, etc. to correspond to the desired shape of the article. In addition, the pockets can have a divider to produce a seal in an intermediate portion of the article, for example, to form a seal or divider as shown at 22″ in FIG. 3. Also, multiple pockets can be provided along the axial length of the rolls 220 so that plural rows of articles can be formed at the same time.

The layers 12, 14 can be single or multiple ply layers which are paid-out from supply spools 226, 228. If the layers are multiple ply layers, they can be stored as multiple ply layers upon the spools 226, 228, or additional plies can be associated in-line by providing additional supply spools as schematically shown in broken at 230, 232. Depending upon the materials of the various substrate plies, the rolls 220 can be heated so that the materials of the layers are welded or fused together as they pass through heated nip rolls 220. Alternately, an adhesive can be applied to the substrates, and the adhesive can be activated by heated nip rolls 220. As a further alternative, a hot or curable adhesive can be applied, with nip rolls 220 (which need not be heated) serving to press the substrate plies together to ensure that, upon cooling or curing, the adhesive provides a satisfactory seal. As discussed earlier, the cosmetic composition(s) can be associated with the substrate by various methods not shown in FIGS. 7A and 7B. For example, a solution containing the composition can be sprayed onto one or both layers or selected portions thereof, with the composition then dried. Alternately, one or both of the layers can be dipped in a solution of the composition and then dried. The cosmetic composition can be applied to the substrate layer(s) before they are stored upon spools, or after the layer(s) are fed from the spools. As a further alternative, the composition can be sandwiched between plies of spools 226, 230 and/or 228, 232, with the plies fastened together to hold the composition(s) therebetween. In this case, it is to be noted that the location of the spools and feed paths of the plies are represented schematically. In particular, if the composition(s) is to be sandwiched between plies, one of the plies is preferably held sufficient horizontal upon application of the composition and adhesive and fastening of the plies to avoid spillage of the composition(s). If the layer 12 and/or 14 is formed of two or more plies, the plies of the respective layer can be coupled together upon passing through rolls 220, or additional rolls can couple the plies of a layer before the layers are coupled together. As a further alternative, the composition(s) can be sandwiched between plies of the layer 12 and/or 14 before being stored on spools 226 or 228.

FIG. 7B depicts an alternative to fastening of the substrate layers by nip rolls 220. In the FIG. 7B arrangement, a reciprocating seal member 240 presses the substrate S against an anvil or back-up member 242 to fasten the substrate layers 12, 14 about the liquid containing capsule or capsules. The seal member 240 includes a recess or aperture 244 to avoid breaking of the liquid containing capsule or capsules. Similarly, the anvil 242 can also have a recess in addition to or in lieu of the recess 244. As with the FIG. 7A arrangement, the seal member 240 can weld the layers together, or it can activate a heat activatable adhesive. Alternately, the seal member 240 can press the layers together to ensure good contact between the seal members to ensure a proper seal is formed by a previously applied adhesive (e.g., a curable or a hot melt adhesive) so that a desirable seal is formed upon cooling or curing of the adhesive. As noted earlier, it is also to be understood that various other expedients can be utilized for joining the substrate layers and plies together. For example, the layers can be stitched or other fastening expedients can be used. As with the FIG. 7A arrangement, plural stamping members 240 can be provided to produce plural articles simultaneously. Also, a divider can extend across the recess or aperture 244 to provide an addition seal or seam as shown at 22″ in FIG. 3.

The liquid containing capsule or capsules can be deposited on the layer 12 by the suitable hopper or dispensing assembly 222. Alternatively, liquid containing capsules can be previously associated with a web which can then be paid-out from a spool as shown, for example, at 230, so that the web having capsules fixed thereto is inserted between the layers 12, 14. If desired, the cosmetic composition(s) could also be deposited between the layers 12, 14. After the layers 12, 14 are fastened together, the individual articles can then be cut from the web by a suitable cutting mechanism (not shown).

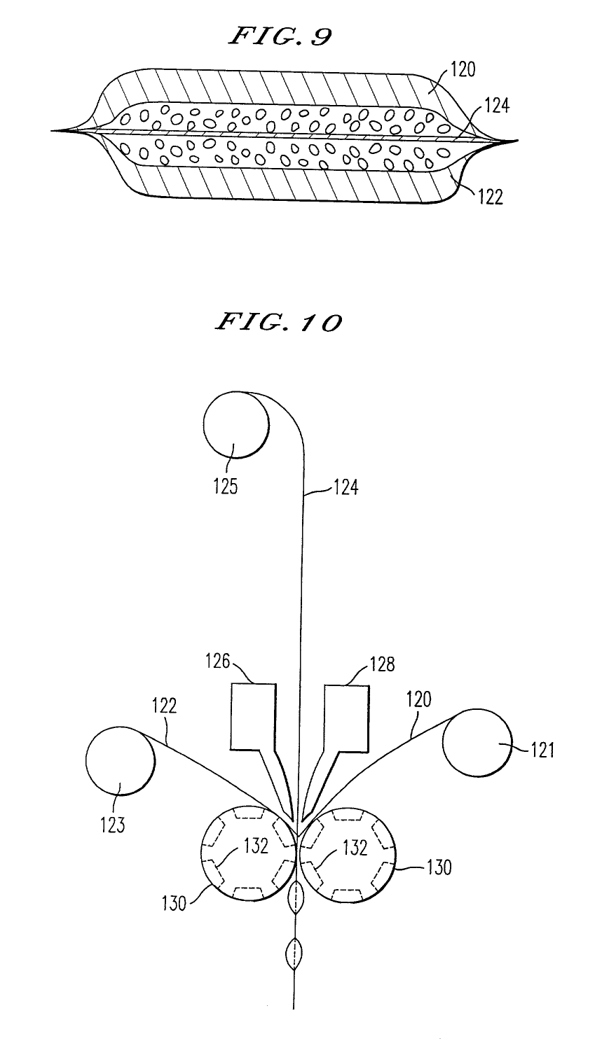

FIGS. 8a-8c depict examples of various webs which can be utilized with the FIGS. 7A-7B method. In the FIG. 8a arrangement, a one ply web 90 forms one layer 12 of the substrate, while another one ply web 92 forms the other layer 14 of the substrate. The liquid capsule or capsules are disposed between the layers and the edges about the substrate are fastened to enclose the microcapsules. The layers 90, 92 of FIG. 8a can be the same, or they can be different so that different sides of the article have different properties.

The layers 12, 14 of FIG. 8b are each two ply layers. With this arrangement, each layer can have a surfactant or other cosmetic composition sandwiched between the plies of that layer. Alternately, a cosmetic composition such as a surfactant could be sandwiched between only one of the layers, with the other layer free of a cosmetic composition. For example, if it is desired to have a wash and dry article as discussed earlier with reference to FIG. 3, a surfactant can be sandwiched between plies of one layer 12, each of which is permeable. The other layer 14 can include an impermeable layer such that when the capsules are broken, the water is not freely dispersed to the outermost ply 100 and the ply 100 can be used for drying the skin. As a further alternative, one or both of the inner plies 96, 98 can be formed of an absorbent material to assist in retaining the moisture within the article after the water capsules are broken. If both inner plies 96, 98 are moisture absorbent, the overall article has good moisture retention properties. If the ply 96 is moisture absorbent and the ply 98 is a moisture barrier, the layer 12 retains moisture and is advantageous for cleansing with a surfactant or application of another cosmetic composition, and the layer 14 is suitable for wiping or drying. As noted earlier, with reference to FIG. 3, if desired, a surfactant (or other composition) can be associated with only part of the layer 12, such that part of the layer 12 is utilized for cleansing (or application of another cosmetic composition) and another part is utilized for rinsing.

As noted above, the surfactant or other composition can be sandwiched between one or both of the layers 12, 14 in the FIG. 8b arrangement. Of course, the surfactant can also be applied to one or both of the layers 12, 14 utilizing other methods as discussed earlier (applying the surfactant as a solution and thereafter drying, etc.). The four plies can be joined together at the same time the layers 12, 14 are sealed about the liquid capsule(s), for example, using four substrate ply supplies as shown at 226, 228, 230, 232 in FIG. 7A. Alternately, the respective plies of one or both of the layer 12, 14 can be joined together before the layers 12, 14 are joined together, with the layers 12, 14 then sealed together about the periphery of the liquid capsule(s) to enclose the liquid capsule(s). Whether the respective plies of each layer are joined prior to or at the same time as the layers 12, 14 will depend upon a number of factors, including the materials of the respective plies and the means selected for attaching those materials, the equipment utilized for coupling the layers together, whether the surfactant or other composition is to be sandwiched between plies or applied with another method, etc.

In the embodiment of FIG. 8c, one of the layers includes two plies, while the other layer includes one ply. This arrangement can be utilized if it is desirable to have a cosmetic composition sandwiched between two plies of one layer, or if it is desirable to have an inner ply of one layer with advantageous moisture absorbent qualities and/or an inner ply of one of the layers as a moisture barrier or moisture impervious layer. As with the FIG. 8b embodiment, the plies 102, 104 can be coupled before or after the layers 12, 14 are coupled together. The plies 102, 106 can be the same or different. For example, the plies 102, 106 can have different textures or roughnesses so that the user can select a preferred side for use. The plies 102, 106 could also have different permeability characteristics, for example, with the ply 106 impermeable or less permeable than layer 102, so that the liquid can be conserved for use in cleansing or applying a cosmetic composition with the play 102 and the liquid is not wasted by passing through the layer 106.

As should be apparent from the foregoing, the substrate can have various forms. Although the substrate layers have been generally depicted as including one or two plies, it is to be understood that one or both layers of the substrate could include more than two plies or layers. For example, it might be desirable to have three or more plies for a given layer. The multiple plies can improve the properties of the substrate, for example, by improving the softness and/or absorbency of the substrate. The multiple plies could also be advantageous from a manufacturing standpoint. For example, plies can be selected of materials which readily lend themselves to retaining the surfactant or other ingredients in dry form, while others selected to impart moisture absorbency/retention characteristics to the substrate, and still other plies could be selected as best suited for contact with the skin. In addition, one of the plies can be selected to provide better bonding between two other plies (e.g., with a center ply disposed between and bonding upper and lower plies upon application of heat and/or pressure). In addition, it could be desirable to completely isolate certain ingredients from the user's skin. For example, in accordance with one of the advantageous embodiments of the invention, ingredients can be provided which, when mixed are exothermic. Such exothermic ingredients can advantageously provide a warm or hot article, however, the ingredients might not be suitable or desirable for contact with the skin. Therefore, such ingredients can be completely surrounded by an impervious or impermeable ply or plies. As should be readily apparent, a number of modifications are contemplated within the scope of the above teachings and examples of the invention described herein.

FIG. 9 depicts a still further embodiment of the invention. The FIG. 9 embodiment includes a first layer 120, a second layer 122, and a water impermeable or water resistant layer 124. Capsules 126, 128 are disposed on either side of the impermeable substrate 64. The capsules 126, 128 can have water therein. Alternately, the capsules 126 on one side of the layer 124 can have water therein, while the capsules 128 on the other side of the layer 124 can include a different substance, such as a liquid conditioner, alcohol, and/or an astringent. As a further alternative, mixtures of microcapsules of different liquids (or a liquid and a powder) could be provided on one or both sides of the layer 124. As a further alternative, some or all of the capsules on one side of the layer 124 can include a liquid, while the capsules on the other side of the layer can include a solid material such as a cosmetic powder, or a material which provides an exothermic reaction upon breaking of the capsule (as discussed further herein). This arrangement provides further flexibility in forming wipes for various purposes. For example, one side of the article can have a surfactant associated therewith, while the other side can have a conditioner, vitamin C, vitamin E, anti-acne or anti-wrinkle compositions, a hair dye, etc. Thus, the user can utilize one side of the article for cleansing the face or hair, and then reverse the article for conditioning and/or applying other ingredients. As noted earlier, this separate application can be advantageous in more reliably applying conditioners or other ingredients to the skin as compared with prior wipes in which the conditioner or other ingredients have been mixed with a surfactant. In addition, the separate application of conditioners or other ingredients can allow the user to control the amount of the conditioner or other ingredients which are to be applied to the skin. The user might desire to apply only a light amount of conditioner or other ingredients, to apply the conditioner or other ingredients to only certain skin areas or to not utilize the conditioner or other ingredients. By providing a wipe which allows the conditioner or other ingredients to be applied separately, the user can better control the cleansing and conditioning steps in caring for the skin or hair. This separate application can be carried out with the article of FIG. 9 in which the surfactant and conditioner (or other ingredients) are provided on opposite sides of the article. This separate application can also be carried out utilizing an article as described earlier with reference to FIG. 3, in which one side of the article includes a cleansing portion and another portion for the application of a conditioner or other ingredients, with the opposite side of the article provided for drying the skin. Although the FIG. 9 embodiment includes plural capsules on each side of the layer 124, as with other embodiments, a single larger or small number of larger capsules can be provided on one or both sides of the layer 124.

As discussed earlier, the surfactant or other cosmetic composition can be provided in dry form in the foregoing embodiments. This can be achieved by forming the layer 120 as a multiply layer with the surfactant sandwiched between plies, by applying an aqueous solution to the layer and then allowing the layer to dry, or by providing a hot coating (containing the composition) to the layer such that when the hot coating cools it is associated with the layer in a solid form which is dissolved once the capsules of water or other liquid are broken. A conditioner or other composition can similarly be with the layer 122. Alternately, one or more of the compositions could be enclosed within the microcapsules.

The article of FIG. 9 can also be advantageously utilized where the article includes a composition which is preferably not exposed to the skin. For example, by providing both layers 122 and 124 as impervious layers, the ingredients disposed between the layers 122 and 124 can be isolated from the skin. By way of example, one or more ingredients can be disposed between the layers 122, 124 which will produce an exothermic reaction in order to warm the article. Therefore, the ingredients disposed between the layers 122, 124 can warm the liquid contained in the capsule(s) disposed between the layers 124, 120. With this arrangement, a warm or hot towel can be provided which does not require an appliance for heating the article. The layer 120 can have a surfactant or other composition associated therewith, or it could be free of a cosmetic composition, such that when the capsules are broken, water or another liquid within the capsules between the layers 124, 120 wet the substrate (with the layer 120 permeable), while breaking of the capsules between the layers 122, 124 causes an exothermic reaction to warm the article, however, because the layers 122, 124 can be impermeable, the ingredient(s) causing the exothermic reaction can be prevented from contacting the skin. The ingredient(s) causing the exothermic reaction can take various forms. For example, two different compositions or materials can be provided in different capsules between the layers 122, 124 (with one, both or neither provided in liquid form), with the materials selected such that, when mixed, an exothermic reaction results. Alternately, one of the compositions can be disposed within the capsules (in liquid or solid form), while another composition is disposed between the layers 122, 124, but not in encapsulated form. The capsules between the layers 122, 124 could also contain a liquid such as water, with one or more materials disposed between the layers 122, 124 (encapsulated or not encapsulated) which produce an exothermic reaction when mixed with the liquid, such that upon breaking of the liquid containing capsule(s), the exothermic reaction is produced. By way of example, anhyrdous magnesium sulfate or anhyrdous calcium chloride could be provided which, when mixed with water, will produce heat. The materials which provide an exothermic reaction could also include a combination of materials which are in dry form, but which, when wetted with a liquid such as water are mixed to generate heat. For example, the materials could include acidic anhydrides and basic anhydrides (or salts of the foregoing) which produce an exothermic reaction when mixed in the presence of water. Examples of such materials include phosphorous pentoxide and calcium oxide which when mixed together in the presence of water will form an exothermic reaction.

As should be apparent, various compositions and arrangements are possible for providing an exothermic reaction in the portion of the substrate between impermeable layers 122, 124. If desired, the layer 122 can be a multi-ply layer, with one ply impervious, and another ply (cloth, foam, sponge, woven or nonwoven materials, etc.) suitable for wiping or drying the skin. Accordingly, with the layer 120 permeable, the user can wet or cleanse the skin utilizing the layer 120, while the outermost ply of the layer 122 can be utilized for wiping or drying, and an exothermic reaction is produced between the impervious plies (the innermost ply of layer 122 and the impervious ply or layer 124) to warm the article. The layer 124 could also be formed as a multiple ply layer, for example, with an impervious or impermeable ply facing toward the layer 122 and a non-impervious or impermeable ply facing toward the layer 122. With this arrangement, the ply of layer 124 which faces layer 122 could be used as a moisture or liquid absorbent ply (to hold the liquid upon breaking of the capsule(s) between layers 122 and 124), and/or the layer 124 can be used for holding a cosmetic composition with the composition held in the permeable ply or between the permeable and impermeable plies.

As should be apparent, in accordance with the invention, wipes can be provided which have multiple functions. As a further alternative, rather than providing different portions of a single wipe which are dedicated to different functions (cleansing, conditioning, rinsing, or application of another composition), different types of wipes can provided within the package containing the wipes. Thus, the user can select to use a cleansing wipe or a drying wipe, followed by the use of a conditioning wipe. With this approach, the user can use the wipes as desired and for example, a conditioner wipe (or wipe having another composition) is not wasted when its use is not desired.

The article of FIG. 9 can be formed in a number of ways. By way of example, the article can be formed as two subarticles utilizing an apparatus as shown, for example, in FIGS. 6 and 7A-B, with the two subarticles then joined together. With this method, the layer 124 includes at least two plies, one ply of each subarticle, with the plies of the subarticles joined to form layer 124 and thereby joining the subarticles to form the article of FIG. 9. Alternately, the arrangement of FIGS. 7A-7B can be utilized in which one or more capsules are first deposited upon one layer (120 or 122) and another layer (124) is then laid over the first layer, and the capsules of the other compartment are then deposited on the layer 124. The remaining layer is then overlaid upon the layer 124 and the layers are sealed together. With such an arrangement, it can be preferable to convey the lower layer utilizing a pocketed conveyor so that the capsules are not spilled upon being deposited upon the lowermost layer, overlaying of the intermediate layer, and depositing of the capsules upon the intermediate layer.

FIG. 10 depicts an alternate arrangement which is suitable for forming articles of the invention, including articles as shown in FIG. 9. The arrangement of FIG. 10 is similar to that of FIG. 7A, but it is vertically disposed. In addition, the capsules are associated with the layers at a location near to the location at which the layers are joined together. As shown in FIG. 10, the various layers 120, 122, 124 can be fed from respective supplies 121, 123, 125. The liquid capsules can be inserted by suitable hopper or inserting mechanisms shown at 126 and 128. The webs are joined together as they pass through nip rolls 130. As with the nip rolls 220, the nip rolls 130 can join the substrate layers utilizing heat and/or an adhesive, and the rolls 130 can be heated or unheated depending upon the fastening expedient selected. As with the rolls 220, the rolls 130 preferably are pocketed to accommodate for the capsules, with the pockets shown in the broken line at 132. The pockets 132 accommodate the liquid containing capsules so that they are not broken as the articles pass through the nip rolls 130. Optionally, suitable arrangements can be provided for causing the layers 120, 122 (or 12, 14 in FIG. 7A) to conform to the pockets 132 (or 224). For example, a vacuum can be applied to the pockets to draw the layers into the pockets, or a suitable mechanical assembly can be provided which mates with the pockets 132 to urge the respective layers into the pockets of the rolls. For example, a roller can be provided adjacent to the rolls 130 having projections which mate with the pockets 132, with the substrate layers 120, 122 passing through the nip between the roll 130 and its respective mating roll prior to filling with the capsules by the hoppers 126, 128 and then passing through the nip between the rolls 130. As noted herein, the capsules disposed on either side of the layer 124 can contain the same liquid, e.g., water, or different liquids. Alternately, the capsule(s) disposed on one side of the layer 124 can include a liquid such as water, and the capsule(s) on the other side of the layer can include an ingredient in solid form (e.g. a cosmetic powder, or a material which can cause an exothermic reaction upon breaking of the capsule(s).

The articles depicted in the preceding figures include multiple small capsules or microcapsules containing the liquid to wet the cosmetic composition. The breakable capsules can also include very small capsules sometimes referred to as microcapsules, which can be formed of various expedients. For example, such capsules can be formed by coacervation. The samples of manufacturing methods for such capsules are disclosed, for example, in U.S. Pat. Nos. 3,691,090; 4,460,563; 4,752,496; and 5,051,304; JP 5 285 210 and JP 8 325 117; and FR 2 795 928, which are incorporated herein by reference.

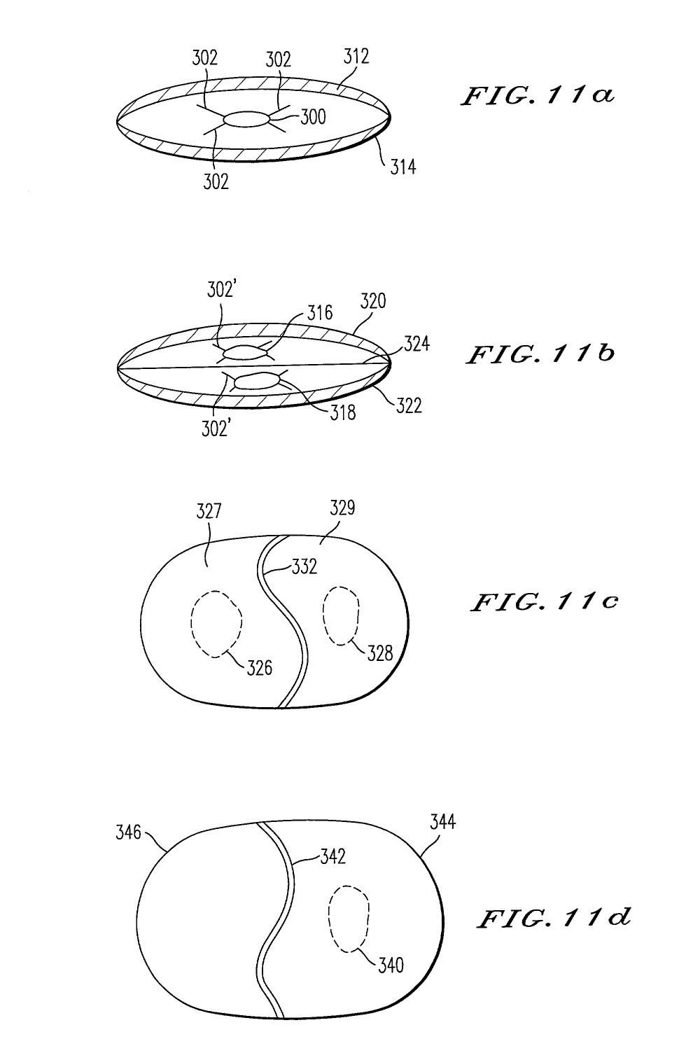

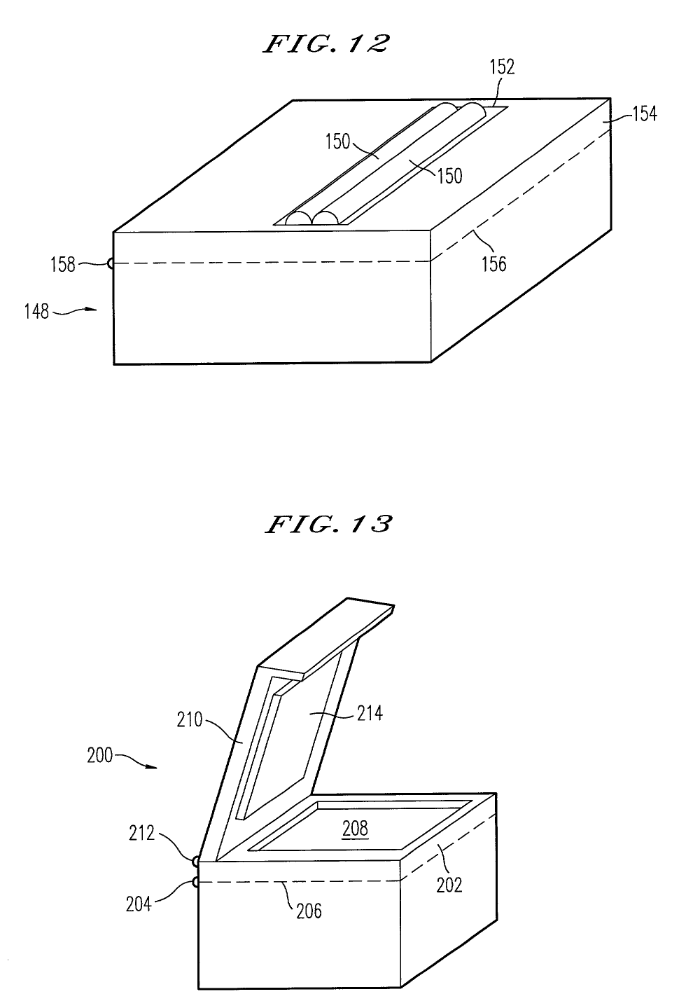

As also noted earlier, the articles can include a single large capsule in lieu of plural smaller capsules. FIGS. 11A-D depict various examples of single capsule arrangements. As shown in FIG. 11A, a single capsule 300 can be disposed between substrate layers 312, 314. As with the previous embodiments, the layers 312, 314 can be formed of the same material. Alternately, the layers 312, 314 can be formed of different materials to provide different texture, roughness, and/or permeability characteristics to the layers. Also, as with the earlier embodiments, the layers 312, 314 can be single ply or multiple ply layers. The capsule 300 of FIG. 11A can optionally include one or more positioning members 302 which extend from the capsule 300. These positioning members can be formed of any suitable material, and preferably are semi-rigid to assist in holding the capsule toward a central region of the article. As shown in FIG. 11A, the positioning members 302 need not be fastened to either of the layers 312, 314, but nevertheless, by projecting from the capsule 300, they inhibit or prevent movement of the capsule 300 so that it is maintained toward a central region of the article. Thus, although the capsule could move, the movement is prevented sufficiently such that the capsule does not become located at an edge of the article. Of course, the supports 302 could be attached to one or both of the layers 312, 314, but this attachment can complicate the manufacturing process. As a further alternative, the capsule could be associated with an intermediate layer which extends between the layers 312, 314 with the capsule 300 fastened (e.g., by an adhesive) to the intermediate layer to hold the capsule in place.

FIG. 11B depicts a two-pocket article, each having a single capsule 316, 318. As with the article of FIG. 11A, the capsules 316, 318 can optionally include supports 302′ to inhibit movement of the capsules. Thus, the arrangement of FIG. 11B is similar to that of FIG. 9, but with the different pockets including only one capsule. Of course, if desired, one pocket could include a single capsule, while another pocket could include plural capsules. The layer 324 corresponds to the layer 124 of FIG. 9, and as with the layer 124 of FIG. 9, the layer 324 is preferably impermeable to separate the pockets. The layers 320, 322 respectively correspond to the layers 120, 122 of FIG. 9, and as with the FIG. 9 embodiment, the layers 320, 322 can have various forms, and it can be single ply or multiple ply layers. As should be apparent, the article of FIG. 11B includes a first pocket between the layers 320, 324, and a second pocket between the layers 324, 322. As with the earlier embodiments, the articles of FIGS. 11A and 11B can include a liquid such as water in the capsule 300 or 316. Also, a cosmetic composition can be disposed such that it is wetted upon breaking of the capsule 300 or 316, and the cosmetic composition can be associated with the substrate by various means as discussed earlier (by locating the composition between layers of the substrate or by associating the composition with one or more of the layers by dipping, spraying, sandwiching the composition between plies of a substrate layer, etc.). Also, as with the embodiments discussed earlier, the capsule 318 of FIG. 11B can include a liquid which wets another cosmetic composition which is associated with the layer 322 or the layer 324, or between the layers 322, 324. Alternately, the capsule 318 could include a cosmetic composition in solid form (such as a make-up powder, rouge, baby powder, etc.). Alternately, the capsule 318 can include water or another composition such that when the capsule 318 is broken and exothermic reaction is produced as discussed earlier. As noted earlier, an article as shown in FIG. 11B can provide various multiple functions. For example, the layer 320 can be used to apply a surfactant while the side of the article 322 can be used for rinsing and/or the application of a conditioner. Alternately, the layer 320 could be used for application of a foundation make-up (e.g., with the liquid in capsule 316 wetting a foundation make-up present in solid form associated with layer 324 and/or 320, or disposed between the layers), with the layer 322 utilized for application of a different cosmetic composition, such as a make-up powder (e.g., with the make-up powder disposed inside of capsule 318). As should be readily apparent, the articles of the invention can have a wide variety of uses. Additional cosmetic compositions which could be utilized can include shampoos, conditioners, hair dyes, anti-acne, anti-wrinkle, and/or vitamin compositions.

FIG. 11C depicts a top view of a two pocket article. In contrast to the arrangement of FIG. 11B, the pockets are side-by-side rather than top and bottom. The capsules are shown in a broken line at 326, 328 in FIG. 11C. Thus, the article of FIG. 11C can be utilized for applying two different compositions with the two different portions of the substrate 327, 329. As with the FIG. 11B embodiment, one portion could include a single capsule while another portion could include multiple capsules if desired. As with the embodiments discussed earlier with reference to FIGS. 1-3, the article can include top and bottom layers which can be the same or different, and one or both of the layers can be formed of single or multiple plies. Preferably, a seam or divider is provided as shown at 332, corresponding to the divider 22″ shown in FIG. 3. Also, as with the FIG. 3 embodiment, the article of FIG. 11C can be utilized for applying two different compositions, or one for applying a composition and the other for rinsing or wiping (i.e., with a liquid capsule provided at that portion of the substrate, but without a cosmetic composition). In addition, as with the FIG. 3 embodiment, the bottom layer of the substrate can include two plies, with one ply adjacent to the capsules being impermeable so that an outermost ply of the bottom layer can be kept dry and utilized for wiping or drying. Thus, as with the FIG. 3 embodiment, the arrangement of FIG. 11C includes a divider 332 which is disposed between first and second portions of the substrate layers, such that a first pocket is provided between the first portion of a first or top substrate layer and a first portion of a second or bottom substrate layer, with a second pocket provided between respective second portions of the first and second (top and bottom) substrate layers.

FIG. 11D depicts yet another arrangement in which a liquid capsule 340 is provided for one portion 344 of the article, while the other portion 346 of the article does not include a liquid containing capsule. The two portions 344, 346 can be separated by a seam or divider as shown at 342. Thus, with the article of FIG. 11D, a pocket is formed between a first or top substrate layer and a second or bottom substrate layer in the portion of the article 344. Further, the seam or divider 342 separates the pocket from a remaining portion 346 of the substrate, and the portion 346 can be utilized for holding the article during application of a cosmetic composition from the portion 344. As with the preceding embodiments, the article of FIG. 11D can include various combinations of single and multiple ply upper and lower layers. The portion 344 can have a cosmetic composition associated therewith as with the earlier embodiments, while the portion 346 can be free of a cosmetic composition. Alternately, the portion 346 could have, e.g. a powder associated therewith. Where multiple side-by-side pockets or sections are provided, the divider or separator seal (22″, 332, 342) can be straight. Alternately, as shown in FIGS. 11C and 11D, the seam or seal can be curved to better match the manner in which the article would be grasped by the user and to provide a better aesthetic appearance.

Preferably, water is encapsulated in the capsules of the various embodiments of the invention so that the cosmetic compositions are wetted upon rupture of the capsules. However, other liquids could be present in some or all of the capsules, such as oils, alcohols, astringents, dyes, etc. The amount of cosmetic composition will vary depending upon the particular composition or compositions selected. In accordance with the articles of the invention, in general, it is preferable to have the compositions provided such that a ratio, by weight, of the liquid to the composition is in the range of 0.3:1 to 50:1. A make-up removal composition would be at the lower end of this range. For example, preferably a make-up removal composition will be in the range of 0.3:1 to 1:1 in terms of the ratio of the liquid to the composition weight. For surfactants, conditioners, and make-up products, the range will be from 1:1 to 50:1 in terms of the ratio of the liquid weight to the cosmetic composition weight. For cleansing compositions, the ratio will liquid to cosmetic composition will preferably be 1:1 to 10:1. Certain compositions could be much higher. For example, with vitamin compositions, the ratio of the liquid to the composition can be as high as 1000:1 by weight.

Also, as noted earlier, the capsules can include one or more compositions which will cause an exothermic reaction upon breaking of the capsules. In addition to the cosmetic compositions discussed herein, the articles can also include an effervescent material, preferably in solid form. The term “solid” is used broadly herein to refer to materials or compositions which are not in liquid form at room temperature, such as powders, particles, granules, or materials which can be present in cake-form. The size of the particles or solid material can depend upon various factors, such as the speed with which it is desired to dissolve the material when wetted. The effervescent can be desirable in that the amount of liquid available is limited because the liquid is contained within one or more of the breakable capsules of the article. Particularly if the article is to include a surfactant, it can be desirable to have good lathering properties for the surfactant, which can be difficult if the liquid supply is limited. The presence of an effervescent material can enhance the lathering ability of the surfactant. As noted herein, the surfactant can take various forms, including soap and non-soap surfactants. Soaps can be desirable due to the lathering ability. Non-soap surfactants typically do not lather as much, they can be beneficial in that they do not typically dry the skin as much as soaps. Thus, for example, in accordance with one example of the invention, the surfactant composition can include a soap and a non-soap surfactant, and in addition, an effervescent material can be provided to enhance the lathering of the article. Of course, the effervescent material could also be provided with other cosmetic compositions as desired. The effervescent material could be provided in powder form, as granules, etc. If desired, the effervescent material could be disposed inside of one or more capsules, so that the effervescent material is not degraded by ambient moisture before use of the article is desired. A powder-like form can be beneficial for the effervescent material if a rapid foaming is desired. By contrast, larger granules (or one or more small cake-sized portions) could be provided for a more prolonged foaming experience. The capsules could also include aqueous solutions or mixtures of water and other ingredients. Further, some capsules could have a solid composition (such as a cosmetic powder which is to be wetted with a liquid or which is used without wetting) or material therein while others have a liquid.

As should be apparent from the foregoing examples, the capsules can vary in size and number and can range from a single large capsule to a plurality of microcapsules. Such capsules are known, and can be formed to break upon application of various amounts of pressure. For example, the capsule(s) can be formed such that they are broken when the article is squeezed in the user's hands, or they could be formed to be broken upon the application of a somewhat more delicate amount of force, such as when pressed upon the skin in use. Generally, a slightly stronger capsule would be desired so that the capsules are not inadvertently broken during handling before use is desired. Where capsules are utilized which break upon application of a more delicate amount of force, suitable packaging should be provided to prevent inadvertent rupture.

The articles could be individually packaged, or packaged as a group within a container. If individually packaged, the user could squeeze the article within the package, and then remove the article after the capsules have been broken. Individual packaging is not required, and a group of articles could be packaged together without individual packaging. Breaking of the capsules within an individual package could be desirable to allow a predetermined amount of mixing time for the liquid within the capsule and the other compositions before the article is removed for use. For example, for certain make-up or hair care compositions, it can be desirable to allow sufficient mixing to ensure a more uniform consistency of the composition. Also, if the article includes a composition to produce an exothermic reaction, the user can be instructed to squeeze the package to mix the ingredients causing the exothermic reaction, and then to wait a predetermined period of time to allow warming of the article. Of course, even where the articles are individually packaged, a number of such individually packaged articles can be packaged and sold within a larger container which protects from inadvertent breakage of the capsules. The individual packages could also be formed to protect the articles from inadvertent breakage of the capsules, in which case the capsules are broken after removal from the package. For example, individual packages can be reinforced with paperboard supports. Two larger individual packages could also be provided, in which the outer layer or outer package is protected from inadvertent breakage, while the capsules can be broken through the inner layer or inner package after removal from the outer package. Group packages can be formed of various rigid or semi-rigid materials including plastics, paperboard or cardboard. Group containers could also be formed of flexible materials, particularly if the group or outer package contains individually packaged articles in which the individual packages are sufficiently protective. Depending upon the size of the article, the cleansing articles of the invention can be packaged in a folded condition. Where the articles are to have a wet side and a dry side (e.g., as in FIG. 3), it can be preferable to fold the article such that the side which is to be wetted is folded upon itself, for example, utilizing a C-fold or a U-fold. With this arrangement, the user can break the microcapsules while the article is folded, and the wet sides are facing each other such that water (or other liquid) is not lost during the breaking process.

As noted earlier, the capsules of the wipes can be broken utilizing hand pressure or upon contact with the skin. Alternatively, according to a further aspect of the invention, the articles can be packaged in containers which include a mechanism for breaking the capsules. FIG. 12 illustrates an example of such a container 148. In this arrangement, rolls 150 are disposed adjacent to a slot 152 so that when the user pulls the article through the nip between the rolls, the capsules are broken. The container could alternately include a non-rollered slot or aperture sized such that when the articles are pulled through the slot, the capsules are broken. However, a roller arrangement is presently preferred over a simple slot, because a non-rollered slot could be difficult to use and could adversely affect the wipe due to friction between the article and the slot. The roller arrangement could be provided at a location through which the articles are dispensed. For example, the articles (or their individual packages) could be interlinked, e.g., in a manner similar to that in which boxes of tissues are linked with one another or by linking the articles (or their packages) by a separatable seam such as a line of perforation. When a wipe is removed from the container, passing the wipe through the rolls causes pressure to be exerted upon the wipe to break the capsules, and the wipe is ready for use. Alternately, the roller arrangement could be provided on an openable cover of the wipe container 152. As shown in FIG. 12, the container 148 can include a lid 154 that is separated from the remainder of the container as indicated by broken line 156. The lid can be hinged as represented at 158 such that the user can open the lid of the container, remove a wipe, and then feed the wipe through the rolls disposed in the lid to break the capsule(s). The wipe is then ready for use.

FIG. 13 depicts an alternate container structure. This container includes an arrangement for compressing the articles within the lid of the container or, more particularly, within a double lid structure. The container 200 includes a lid 202 which is hinged to the remainder of the container as represented at 204. The lid 202 is shown in the closed position, with line 206 indicating the separation between the lid 202 and the remainder of the container. The lid 202 includes a compartment or recess 208, and an additional lid 210 is hingedly mounted upon lid 202 as represented at 212. To use this container, the user opens the lid 202 to remove an article from the main or first compartment of the container. The main compartment of the container holds at least one, and preferably multiple articles for storage and to prevent breakage of the capsules of the articles while the articles are stored therein. After the user removes an article from the main compartment, the user then opens the second lid 210 and places the article within the compartment 208. This compartment is sized such that when the lid 210 is closed, the article is compressed to break the capsule(s). Optionally, the lid 210 can include one or more projections which extend into the compartment 208 to further assist in compressing the article, and/or one or more projections can be provided in the bottom of the compartment 208. One large projection is shown at 214 in FIG. 11. One or more smaller projections could also be provided. Particularly if the wipe is to include a single capsule, a sharper projection could be desirable to provide a concentrated breaking or piercing force to break the capsule. Although the lid 210 and compartment 208 are associated with the top of the lid 202 in the FIG. 11 arrangement, such a compression lid and compartment could be disposed at other locations, for example, along the walls on the inside or the outside of the container, or on an inside portion of the lid 202. The provision of a device for breaking the capsules can be advantageous in a number of respects. For example, if the articles include a large number of small capsules, a breaking mechanism can ensure a greater quantity of the capsules are broken, thereby making more effective use of the liquids enclosed within the capsules. By contrast, with manual breaking methods, some of the capsules can escape the compressive forces and remain unbroken, or the user might excessively squeeze the article which could result in squeezing the moisture from the article or otherwise diminish the performance of the article. Also, a breaking mechanism can be advantageous for single or multiple capsule arrangements, because the capsule could then be formed to break with a higher breaking pressure or force, so that inadvertent breakage during manufacture or subsequent handling can be minimized. The compression device can be sized and shaped to compress the articles while in individual packages or individual packages. Also, the compression device can be sized to break capsules of the articles in either folded or unfolded conditions as desired.

A wide variety of materials can be used for the substrate layers or plies of the substrate layers. Nonlimiting examples of suitable substrates include natural or synthetic nonwoven or woven substrates, natural sponges, synthetic sponges or foam materials, and combinations of the foregoing. Nonwoven substrates are desirable because they are economical and readily available in a variety of materials with a variety of properties. Nonwoven materials include synthetic and/or natural fibers which are not woven into a fabric but rather are formed into a sheet, mat, or pad layer. The fibers can either be randomly aligned or they can be carded, i.e., combed to be oriented in primarily one direction. Furthermore, nonwoven substrates can include a combination of layers of random and carded fibers. Substrates can also be formed by hydroentanglement, air entanglement or other known methods. By way of example, a variety of substrate materials are available from Polymer Group Incorporated (PGI), and other vendors such as DuPont, Norafin, Tharreau Industries, Jacob-Holm Industries, or BBA Nonwovens. Various nonlimiting examples of such substrates include Duralace 7006, Duralace 7123, Duralace 9796, Sontara 8021, Sontara 8801, Sontara 9951, Sontara 9957, (with the Sontara line available from DuPont), Norafin 1.73065.01, Aquadim VE 50 L (from Tharreau), Lifast 55, ref 321055 (from Jacob-Holm), and BBA Ultraloft 182-010, Flexilon 140-130, and Novonette 149-807. Various sponge or foam like materials can also be used as discussed herein. Sponge or foam-like materials can be advantageous for their softness and moisture retention capabilities (which can be particularly desirable when using an encapsulated liquid because the amount of liquid available is limited). The moisture retention capabilities of a sponge or foam material can also be desirable, e.g., where the article includes ingredients which produce an exothermic reaction so that the sponge or foam material can retain water or other liquids while they are warmed. For example, as discussed earlier, with the FIG. 9 article in which an exothermic reaction occurs between layers 122 and 124, the layer 120 can include a sponge or foam materials, or another absorbent material so that the water or other liquids from the capsules between layers 124, 120 are retained while being warmed. The layer 120 could additionally include an outer ply disposed over the absorbent ply, with the outer ply having properties more desirable for contact with the skin. The substrate layers or plies could also include various natural materials such as paper or cotton-based substrates.

Examples of lathering surfactants include anionic lathering surfactants such as ammonium lauroyl sarcosinate, sodium trideceth sulfate, sodium lauroyl sarcosinate, ammonium laureth sulfate, sodium laureth sulfate, ammonium lauryl sulfate, sodium lauryl sulfate, sodium myristate, ammonium cocoyl isethionate, sodium cocoyl isethionate, sodium lauroyl isethionate, sodium cetyl sulfate, and mixtures thereof, nonionic lathering surfactants including lauramine oxide, cocoamine oxide, decyl polyglucose, lauryl polyglucose, sucrose cocoate, C12-14 glucosamides, sucrose laurate, and mixtures thereof, amphoteric lathering surfactants including disodium lauroamphodiacetate, sodium lauroamphoacetate, cetyl dimethyl betaine, cocoamidopropyl betaine, cocoamidopropyl hydroxy sultaine, and mixtures thereof.

As noted earlier, various additional ingredients could also be provided with the surfactant, including conditioners, vitamins, anti-wrinkle agents, sunscreens, etc. As also noted earlier, in accordance with one of the advantageous aspects of the invention, because the water is associated with the substrate, problems associated with excessive water (for example, with prior dry wipes in which the wipe is held for an excessive period of time beneath a faucet) can be avoided. These additional ingredients can be combined with the surfactant, or applied to a different portion of the article so that the additional ingredients can be separately applied by the user.

What is claimed is:

1. A cosmetic article comprising:
(a) a substrate;
(b) a cosmetic composition associated with said substrate;
(c) at least one breakable capsule associated with said substrate, said at least one breakable capsule containing a liquid;
wherein upon breaking of said at least one breakable capsule, said liquid wets said cosmetic composition.

2. A cosmetic article as recited in claim 1, wherein said substrate includes a first layer and a second layer, and wherein:
at least one of said first layer and said second layer is permeable to said liquid; and
said cosmetic composition is associated with said first layer.

3. A cosmetic article as recited in claim 2, wherein at least part of said second layer is impermeable to said liquid.

4. A cosmetic article as recited in claim 2, wherein said at least one breakable capsule is disposed between said first layer and said second layer.

5. A cosmetic article as recited in claim 4, wherein said first layer comprises a first ply and a second ply, and wherein said cosmetic composition is at least partially disposed between said first ply and said second ply.

6. A cosmetic article as recited in claim 4, wherein said at least one breakable capsule contains water.

7. A cosmetic article as recited in claim 6, wherein said cosmetic composition includes a surfactant.

8. A cosmetic article as recited in claim 1, wherein said substrate contains a plurality of said breakable capsules.

9. A cosmetic article as recited in claim 8, wherein said liquid comprises water.

10. A cosmetic article as recited in claim 1, said cosmetic composition includes a surfactant, and wherein a ratio of said liquid to said cosmetic composition is in the range of 1:1 to 10:1 by weight.

11. A cosmetic article as recited in claim 1, wherein said substrate comprises:
(i) a first portion having a surfactant associated therewith; and
(ii) a second portion having an amount of said surfactant less than said first portion;
and wherein said at least one breakable capsule comprises:
(iii) a first breakable capsule disposed adjacent to said first portion of said substrate for wetting said first portion; and
(iv) a second breakable capsule adjacent said second portion of said substrate for wetting said second portion.

12. A cosmetic article as recited in claim 11, wherein said second portion is substantially free of said surfactant.

13. A cosmetic article as recited in claim 11, wherein said substrate comprises a first layer and a second layer, and wherein said first layer includes said first and second portions of said substrate.

14. A cosmetic article as recited in claim 13, wherein said first portion of said substrate comprises indicia indicating said first portion of said substrate is for cleansing.

15. A cosmetic article as recited in claim 14, wherein said second portion of said substrate comprises indicia indicating said second portion of said substrate is for rinsing.

16. A cosmetic article as recited in claim 14, wherein a conditioner is associated with said second portion of said substrate, and said second portion comprises indicia indicating second portion of said substrate is for conditioning.

17. A cosmetic article as recited in claim 13, wherein said second layer of said substrate comprises a first impermeable ply and a second ply, and further wherein said first impermeable ply is disposed between said at least one breakable capsule and said second ply such that said second ply of said second layer remains substantially dry upon breaking of said at least one breakable capsule.

18. A cosmetic article as recited in claim 17, wherein said second ply comprises indicia indicating said second ply is for drying.

19. A cosmetic article as recited in claim 11, wherein said first portion of said substrate comprises indicia indicating said first portion is for cleansing, said second portion of said substrate comprises indicia indicating said second portion is for conditioning, and wherein said substrate further comprises a third portion, and wherein said third portion of said substrate comprises indicia indicating said third portion is for drying.

20. A cosmetic article as recited in claim 11, wherein said first portion of said substrate comprises indicia indicating said first portion is for cleansing, said second portion of said substrate comprises indicia indicating said second portion is for rinsing, and wherein said substrate further comprises a third portion, and wherein said third portion of said substrate comprises indicia indicating said third portion is for drying.

21. A cosmetic as recited in claim 11, wherein said first portion of said substrate is a first substrate layer and said second portion of said substrate is a second substrate layer, said cleansing article further comprising;
a third substrate layer disposed between said first substrate layer and said second substrate layer, wherein said first breakable capsule is disposed between said first layer and said third layer, and wherein said second breakable capsule is disposed between said second layer and said third layer, and wherein said third layer is an impermeable layer.

22. A cosmetic article as recited in claim 21, wherein said first portion comprises indicia indicating said first portion is for cleansing.

23. A cosmetic article as recited in claim 22, wherein a conditioner is associated with said second portion of said substrate such that said conditioner is wetted when said second breakable capsule is broken, and wherein said second portion comprises indicia indicating said second portion is for conditioning.

24. A cosmetic article as recited in claim 21, wherein said first substrate layer comprises a first ply and a second ply, and wherein said surfactant is at least partially disposed between said first ply and said second ply.

25. A cosmetic article as recited in claim 1, wherein said cosmetic composition is a make-up removal composition, and wherein a ratio of said liquid to said make-up removal composition is less than or equal to 1.0 by weight.

26. A cosmetic article as recited in claim 23, wherein said ratio is greater than or equal to 0.3 by weight.

27. A cosmetic article as recited in claim 1, wherein a ratio of said liquid to said cosmetic composition is in the range of 1:1 to 50:1 by weight.

28. A cosmetic article as recited in claim 27, wherein said cosmetic composition includes at least one of: a make-up composition, a surfactant composition, and a conditioner.

29. A cosmetic article as recited in claim 1, wherein said cosmetic composition includes a surfactant, and wherein a ratio of said liquid to said surfactant in the range of 1:1 to 10:1 by weight.

30. A cosmetic article as recited in any one of the preceding claims, further comprising a container within which at least one cosmetic article is disposed.

31. A cosmetic product comprising:
(a) a container;
(b) at least one cosmetic article disposed in said container, said at least one cosmetic article comprising:
(i) a substrate;
(ii) at least one breakable capsule associated with said substrate, said at least one breakable capsule including a liquid such that upon breaking of said at least one breakable capsule said liquid wets said substrate; and
(c) wherein said container includes means for breaking said at least one breakable capsule.

32. A cosmetic product as set forth in claim 31, wherein said means for breaking said at least one breakable capsule comprises:
an opening disposed in said container, wherein upon passing of said cosmetic article through said opening, said cosmetic article is compressed to break said at least one breakable capsule.

33. A cosmetic product as recited in claim 32, further including a pair of rolls disposed adjacent to said opening.

34. A cosmetic product as recited in claim 31, wherein said means for breaking said breakable capsules comprises:
(i) a compartment;
(ii) a compartment lid disposed adjacent to said compartment; and
(iii) wherein said compartment is sized such that upon placement of the cosmetic article in said compartment and closing said lid, said at least one breakable capsule is broken.

35. A cosmetic product as recited in claim 34, wherein said container includes an article storage compartment for storing said plurality of said cosmetic articles and a container lid which selectively opens and closes said article storage compartment, and further wherein said compartment for breaking said at least one breakable capsule is disposed in said container lid.

36. A cosmetic product as recited in claim 31, wherein said plurality of skin care articles includes a first plurality of articles and a second plurality of articles, and wherein said first and second pluralities of articles include at least one of a surfactant and a conditioner;
wherein a ratio of surfactant weight to conditioner weight in said first plurality of articles is in a range of 0.0 to 1.0; and
wherein a ratio of conditioner weight to surfactant weight in said second plurality of articles is in a range of 0.0 to 1.0.

37. A cosmetic product as recited in claim 31, wherein said cosmetic article includes a first portion and a second portion, and wherein said first and second portions include at least one of a surfactant and a conditioner, and further wherein a ratio of surfactant weight to conditioner weight in said first portion is in a range of 0.0 to 1.0, and a ratio of conditioner weight to surfactant weight is said second portion is in a range of 0.0 to 1.0.

38. A cosmetic product as recited in claim 31, further including a cosmetic composition associated with said substrate in solid form, and wherein said liquid wets said cosmetic composition upon breaking of said at least one breakable capsule.

39. A cosmetic product as recited in claim 38, wherein a ratio of liquid to said cosmetic composition is in a range of 0.30:1 to 50:1.

40. A cosmetic product as recited in claim 39, wherein said cosmetic composition comprises a make-up removal composition, and wherein a ratio of said liquid to said make-up removal composition is in a range of 0.3:1 to 1:1 by weight.

41. A method of forming a cosmetic article comprising:
providing at least one substrate web;
applying a cosmetic composition to at least a portion of said at least one substrate web;
providing at least one liquid containing capsule; and
fastening edges of said at least one substrate web such that said at least one liquid containing capsule is held within said at least one substrate web.

42. A method as recited in claim 41, providing a plurality of substrate webs and coupling said plurality of substrate webs together to form a composite substrate web before the step of fastening edges to hold said at least one liquid containing capsule.

43. A method as recited in claim 41, wherein said at least one substrate web includes a first substrate ply and a second substrate ply, and wherein the step of applying a cosmetic composition includes sandwiching the cosmetic composition between said first substrate ply and said second substrate ply.

44. A method as recited in claim 41, wherein the step of applying a cosmetic composition includes providing the cosmetic composition in an aqueous solution, and applying the aqueous solution to at least a portion of said substrate, and thereafter drying said at least one portion.

45. A method as recited in claim 41, wherein said at least one substrate web comprises:
at least one permeable substrate ply; and
at least one impermeable substrate ply.

46. A method as recited in claim 41, wherein said at least one substrate web includes a first substrate layer, a second substrate layer, and a third substrate layer, and wherein said at least one breakable capsule includes a first breakable capsule positioned between said first substrate layer and said second substrate layer, and a second breakable capsule between said second substrate layer and said third substrate layer, and thereafter coupling said first substrate layer to said second substrate layer and coupling said second substrate layer to said third substrate layer.

47. A method as recited in claim 46, further including providing a plurality of said first breakable capsules and a plurality of said second breakable capsules.

48. A method as recited in claim 46, wherein said cosmetic composition is associated with one of said first, second and third substrate layers.

49. A method as recited in claim 48, wherein said second substrate layer comprises an impermeable ply.

50. A method as recited in claim 46, wherein at least one of said first, second, and third substrate layers includes first and second plies, and wherein the step of applying said cosmetic composition to at least a portion of said at least one substrate web includes sandwiching said cosmetic composition between said first and second plies.

51. A method as recited in claim 41, wherein the step of providing at least one substrate web includes providing a first substrate web which is at least partially permeable, providing a second substrate web which is impermeable, and providing a third substrate web, and wherein said at least one breakable capsule is positioned between said first substrate web and said second substrate web.

52. A cosmetic article comprising:
a first substrate layer;
a second substrate layer;
a third substrate layer;
a first breakable capsule containing a liquid disposed between said first substrate layer and said second substrate layer;
a second breakable capsule disposed between said second substrate layer and said third substrate layer, said second breakable capsule including a first material, and further wherein a second material is disposed between said second substrate layer and said third substrate layer, and wherein said first material and said second material are selected such that upon breaking of said second breakable capsule an exothermic reaction is produced and said exothermic reaction heats the liquid of said first breakable capsule.

53. A cosmetic article as recited in claim 52, wherein said article further includes a cosmetic composition.

54. A cosmetic article as recited in claim 53, wherein said cosmetic composition includes a surfactant.

55. A cosmetic article as recited in claim 54, wherein said surfactant is associated with one of said first and second substrate layers.

56. A cosmetic article s recited in claim 55, wherein at least one of said first substrate layer and said second layer includes two substrate plies, and further wherein said surfactant is at least partially disposed between said two substrate plies.

57. A cosmetic article s recited in claim 52, wherein a first cosmetic composition is associated with said first substrate layer.

58. A cosmetic article as recited in claim 57, wherein said first cosmetic composition is located in a first portion of said first substrate layer, and wherein said article further includes a second cosmetic composition located in a second portion of said first substrate layer.

59. A cosmetic article as recited in claim 58, wherein a divider extends from a location between said first and second portions of said first substrate layer to said second substrate layer such that a first pocket is disposed between said first portion and said second substrate layer and a second pocket is disposed between said second portion and said second substrate layer, and further wherein said first breakable capsule is disposed in said first pocket and a third breakable capsule is disposed in said second pocket.

60. A cosmetic article as recited in claim 59, wherein said first cosmetic composition includes a surfactant, and said second cosmetic composition includes a conditioner.

61. A cosmetic article as recited in claim 58, wherein said first cosmetic composition includes a surfactant, and said second cosmetic composition includes a conditioner.

62. A cosmetic article as recited in claim 60, further including an effervescent composition which is wetted upon breaking of said first breakable capsule to enhance foaming of said surfactant.

63. A cosmetic article as recited in claim 54, further including an effervescent composition which is wetted upon breaking of said first breakable capsule to enhance foaming of said surfactant.

64. A cosmetic article as recited in claim 52, wherein said second substrate layer includes at a first moisture impermeable ply.

65. A cosmetic article as recited in claim 64, wherein at least one of said first and second substrate layers includes an absorbent ply which retains the liquid of said first breakable capsule.

66. A cosmetic article as recited in claim 65, wherein said absorbent ply includes one of a foam and a sponge material.

67. A cosmetic article as recited in claim 64, wherein said third substrate layer includes a second moisture impermeable ply.

68. A cosmetic article as recited in claim 67, wherein said third substrate layer further comprises an outer substrate layer that can be used for wiping or drying skin, and wherein said second moisture impermeable ply shields said outer ply from said first and second materials.

69. A cosmetic article as recited in claim 68, wherein a cosmetic composition is associated with said first layer.

70. A cosmetic article as recited in claim 68, wherein said first layer includes a first portion and a second portion, and wherein a divider extends from a location between said first portion and said second portion to said second layer to form a first pocket between said first portion and said second layer and a second pocket between said second portion and said second layer, and wherein said first breakable capsule is disposed in said first pocket.

71. A cosmetic article as recited in claim 70, further including a first cosmetic composition in solid form disposed in at least one of said first portion, said second substrate layer, and said first pocket, and wherein said first cosmetic composition is wetted by the liquid of said first breakable capsule upon breaking of said first breakable capsule.

72. A cosmetic article as recited in claim 70, further including a third breakable capsule disposed in said second pocket.

73. A cosmetic article as recited in claim 72, wherein said first cosmetic composition includes a surfactant composition.

74. A cosmetic article as recited in claim 73, wherein said third breakable capsule includes a rinsing liquid.

75. A cosmetic article as recited in claim 74, wherein said surfactant composition comprises a soap and a non-soap surfactant.

76. A cosmetic article as recited in claim 75, further including an effervescent composition which is wetted upon breaking of said first breakable capsule to enhance lathering of said surfactant composition.

77. A cosmetic article as recited in claim 72, further comprising a second cosmetic composition located in at least one of said second portion of said first substrate layer, said second pocket, and a portion of said second substrate layer adjacent to said second pocket, and wherein said third breakable capsule includes a liquid which wets said second cosmetic composition when said third breakable capsule is broken.

78. A cosmetic article as recited in claim 77, wherein said first cosmetic composition includes a surfactant, and said second cosmetic composition includes a conditioner.

79. A cosmetic article as recited in claim 78, wherein water is contained in said first and third breakable capsules.

80. A cosmetic article as recited in claim 72, wherein water is contained in at least one of said first and second breakable capsules.

81. A cosmetic article as recited in claim 80, wherein said article includes a plurality of said first breakable capsules and a plurality of said second breakable capsules.

82. A cosmetic article comprising:
a first substrate layer;
a second substrate layer, wherein said second substrate layer includes at least one moisture impermeable ply;
a third substrate layer;
a first breakable capsule disposed between said first substrate layer and said second substrate layer, said first breakable capsule containing a first liquid;
a first cosmetic composition disposed such that upon breaking of said first breakable capsule, said first cosmetic composition is wetted by said first liquid;
a second breakable capsule disposed between said second substrate layer and said third substrate layer.

83. A cosmetic article as recited in claim 82, wherein said second breakable capsule includes a second cosmetic composition disposed therein.

84. A cosmetic article as recited in claim 83, wherein said second cosmetic composition comprises a powder.

85. A cosmetic article as recited in claim 82, wherein said second breakable capsule includes a second liquid, said article further comprising a second cosmetic composition disposed such that said second liquid wets said second composition upon breaking of said second breakable capsule.

86. A cosmetic article as recited in claim 85, wherein said first cosmetic composition includes a surfactant composition, and said second cosmetic composition includes a conditioner.

87. A cosmetic article as recited in claim 86, wherein said surfactant composition includes a soap and a non-soap surfactant.

88. A cosmetic article as recited in claim 86, further including an effervescent material disposed such that said first liquid wets said effervescent material upon breaking of said first breakable capsule and assists lathering of said surfactant.

89. A cosmetic article as recited in claim 82, further including a third breakable capsule disposed between said first substrate layer and said second substrate layer, said third breakable capsule including an effervescent material in solid form, and wherein upon breaking of said first and third breakable capsules said first liquid wets said effervescent material.

90. A cosmetic article as recited in claim 89, wherein said first breakable capsule contains water.

91. A cosmetic article as recited in claim 90, wherein said article includes a plurality of said first capsules in a plurality of said third capsules.

92. A cosmetic article as recited in claim 82, wherein said first cosmetic composition includes a make-up composition.

93. A cosmetic article as recited in claim 92, wherein said make-up composition includes a foundation make-up.

94. A cosmetic article as recited in claim 93, wherein said second breakable capsule includes a cosmetic powder.

95. A cosmetic article comprising:
a substrate, said substrate including a first pocket and a second pocket;
a first breakable capsule disposed in said first pocket, said first breakable capsule containing a first liquid;
a second breakable capsule disposed in said second pocket; and
a cosmetic composition disposed such that upon breaking of said first breakable capsule, said liquid wets said cosmetic composition.

96. A cosmetic article as recited in claim 95, wherein said first breakable capsule contains water.

97. A cosmetic article as recited in claim 95, wherein said second breakable capsule contains a second liquid.

98. A cosmetic article as recited in claim 95, wherein said first and second breakable capsules contain water.

99. A cosmetic article as recited in claim 98, further including a second cosmetic composition disposed such that upon breaking of said breakable capsule, water of said second capsule wets said second cosmetic composition.

100. A cosmetic article as recited in claim 95, wherein said substrate includes:
a first substrate layer;
a second substrate layer; and
a third substrate layer;
wherein said first pocket is disposed between said first substrate layer and said second substrate layer, and said second pocket is disposed between said second substrate layer and said third substrate layer.

101. A cosmetic article as recited in claim 95, wherein said substrate includes:
a first substrate layer having first and second portions;
a second substrate layer having first and second portions; and
a divider which couples said first substrate layer to said second substrate layer at a location between the respective first and second portions of the first and second substrate layers, such that said first pocket is disposed between the first portion of the first substrate layer and the first portion of the second substrate layer and said second pocket is disposed between said second portion of said first substrate layer and said second portion of said second substrate layer.

102. A cosmetic article as recited in claim 95, further including means coupled to at least one of said first and second breakable capsules to prevent movement of said at least one of said first and second breakable capsules.

103. A cosmetic article comprising:
a substrate having at least one pocket therein, said at least one pocket including a breakable capsule containing a liquid, said article further comprising a cosmetic composition disposed such that upon breaking of said breakable capsule, said liquid wets said cosmetic composition, the substrate further comprising a holding portion for holding said article during use.

104. A cosmetic article as recited in claim 103, wherein said substrate comprises:
a first substrate layer;
a second substrate layer; and
a divider which divides said substrate into a first portion and a second portion, and wherein said at least one pocket is disposed in said first portion and between said first substrate layer and said second substrate layer and further wherein said second portion comprises said holding portion.

105. A cosmetic product comprising:
a container;
a cosmetic article disposed inside of said container, said cosmetic article, including a substrate and at least one breakable capsule disposed inside of said substrate; and
wherein said container includes means for breaking said at least one breakable capsule.

106. A cosmetic product as recited in claim 105, wherein said container comprises:
a first compartment for storing said at least one article;
said first compartment including a first lid; and
wherein said means for breaking includes a second compartment for breaking said at least one breakable capsule, said second compartment including a second lid, and wherein when said cosmetic article is disposed in said second compartment, closing of said second lid breaks said at least one breakable capsule.

107. A cosmetic product as recited in claim 106, wherein said second compartment is disposed inside of said first lid.

108. A cosmetic product as recited in claim 106, wherein said means for breaking includes an aperture extending through said container, and wherein said at least one breakable capsule is broken upon passing of said article through said aperture.

109. A cosmetic product as recited in claim 108, further including a pair of rollers disposed adjacent to said aperture.

110. A cosmetic product as recited in claim 108, wherein said at least one breakable capsule includes a first breakable capsule containing water, and wherein said article further includes a cosmetic composition in solid form such that upon breaking of said first breakable capsule, said water wets said cosmetic composition.

111. A cosmetic product as recited in claim 105, wherein said at least one breakable capsule includes a first breakable capsule containing a liquid and a second breakable capsule, said article further including means for producing an exothermic reaction upon breaking of said second breakable capsule.

112. A cosmetic product as recited in claim 105, wherein said article includes a liquid disposed in said at least one breakable capsule and a surfactant composition, wherein said liquid in said at least one breakable capsule wets said surfactant composition upon breaking of said at least one breakable capsule.

113. A cosmetic product as recited in claim 112, wherein said surfactant composition includes a soap and a non-soap surfactant, and wherein the article further includes an effervescent material in solid form which is wetted upon breaking of said at least one breakable capsule to assist lathering of said surfactant composition.

114. A cosmetic article comprising:
a substrate having at least one pocket therein, said at least one pocket including a breakable capsule containing a liquid, said article further comprising a cosmetic composition disposed such that upon breaking of said breakable capsule, said liquid wets said cosmetic composition.
